SEQUENCE LISTING (16 sequences)

SEQ ID NO: 1 - protein, 13 aa, synthetic construct
  MOD_RES 1: note = Cholic Acid (ChAc)
CGYGPKKKRKVGG

SEQ ID NO: 2 - protein, 7 aa, synthetic construct
PKKKRKV

SEQ ID NO: 3 - protein, 19 aa, synthetic construct
CGWWGYGPKKKRKVGGWWG

SEQ ID NO: 4 - protein, 20 aa, synthetic construct
CSNFGPMKGGNFGGRSSGPY

SEQ ID NO: 5 - protein, 20 aa, synthetic construct
CSGYGKVSRRGGHQNSYKPY

SEQ ID NO: 6 - protein, 20 aa, synthetic construct
CNEKRKEKNIKRGGNRFEPY

SEQ ID NO: 7 - protein, 21 aa, synthetic construct
CADREEGKERRHHRREELAPY

SEQ ID NO: 8 - protein, 34 aa, synthetic construct
CNKRVCEEIAIIPSKKLRNKGSGRIQRGPVRGIS

SEQ ID NO: 9 - protein, 16 aa, synthetic construct
CMGRVRTKTVKKAAGG

SEQ ID NO: 10 - protein, 20 aa, synthetic construct
CNKRVCEEIAIIPSKKLRNK

SEQ ID NO: 11 - protein, 20 aa, synthetic construct
CSKKLRNKIAGYVTHLMKRI

SEQ ID NO: 12 - protein, 15 aa, synthetic construct
CGYGPAAKRVKLDGG

SEQ ID NO: 13 - protein, 22 aa, synthetic construct
CGRFSPMGVDHMSGLSGVNVPG

SEQ ID NO: 14 - protein, 15 aa, synthetic construct
CGYGKLKIKRPVKGG

SEQ ID NO: 15 - protein, 21 aa, synthetic construct
CAVKRPAATKKAGQAKKKKLD

SEQ ID NO: 16 - protein, 8 aa, synthetic construct
SIINFEKL

(19) United States
(12) Patent Application Publication
(10) Pub. No.: US 2025/0049914 A1
(43) Pub. Date: Feb. 13, 2025

(54) STEROID ACID-PEPTIDE BASED INTRACELLULAR CARGO DELIVERY

(71) Applicant: DEFENCE THERAPEUTICS INC., Vancouver (CA)

(72) Inventor: Simon Beaudoin, Sherbrooke (CA)

(21) Appl. No.: 18/717,506

(22) PCT Filed: Dec. 8, 2022
(86) PCT No.: PCT/CA2022/051795

§ 371 Date: Jun. 7, 2024

Related U.S. Application Data
(60) Provisional application No. 63/265,125, filed on Dec. 8, 2021.
(60) Provisional application No. 63/362,487, filed on Apr. 5, 2022.

(51) Int. Cl. A61K 39/385; A61K 38/46; A61K 39/00; A61K 39/215; A61K 47/54; A61K 47/64; A61P 35/00

(52) U.S. Cl. CPC A61K 39/385; A61K 38/465; A61K 39/0011; A61K 39/215; A61K 39/461; A61K 47/554; A61K 47/64; A61P 35/00

(57) ABSTRACT

Described herein are compositions and methods relating to cargoes for improved intracellular delivery. The compositions generally comprise a steroid acid-peptide conjugate covalently linked to and/or admixed with a cargo to be delivered intracellularly to target cells, resulting in increased intracellular and/or cytosolic/nuclear delivery of the cargo, increased presentation of the cargo by target cells, increased intracellular reactive oxygen species production in target cells, and/or the transformation of immunosuppressive cells into immunostimulatory and/or proinflammatory cells. Methods of improving cargo stability via covalent conjugation with steroid acid-peptide moieties are also described herein, as well as the use of steroid acid-peptide conjugates for enhanced genome editing and in the production of cell-based vaccines.

The present description relates to intracellular delivery of biologically active cargoes. More specifically, the present description relates to compositions comprising a steroid acid-peptide conjugate covalently linked to, and/or admixed with, a cargo for improved intracellular, cytosolic and/or nuclear delivery, as well as to the use of such compositions for example in genome editing and the manufacture of cell-based vaccines. The present description further relates to methods of increasing the stability of cargoes via their covalent conjugation to one or more steroid acid-peptide moieties. The present description refers to a number of documents, the contents of which is herein incorporated by reference in their entirety.

### BACKGROUND

Intracellular delivery of biological cargoes such as peptides, proteins, and polynucleotides generally rely on the endocytic pathway as the major uptake mechanism, resulting in a large fraction of the cargoes being trapped inside endosomes/lysosomes. Such trapped cargoes often remain sequestered from their intended targets and may be degraded. Thus, improved strategies for increasing intracellular delivery and avoiding endosomal entrapment would be highly desirable.

### SUMMARY

In a first aspect, described herein is a composition comprising a steroid acid-peptide conjugate covalently linked to, and/or admixed with, a cargo for intracellular delivery. In some embodiments, covalently linking the cargo to the steroid acid-peptide conjugate increases intracellular delivery and/or cytosolic/nuclear delivery of the cargo, as compared to a corresponding composition lacking the steroid acid-peptide conjugate. In some embodiments, the cargo is admixed with a sufficient concentration of the steroid acid-peptide conjugate to increase intracellular delivery and/or cytosolic/nuclear delivery of the cargo, as compared to a corresponding composition lacking admixture with the steroid acid-peptide conjugate. In particular embodiments, the steroid acid may be a bile acid and the peptide may comprise a functional nuclear localization signal (NLS) or other subcellular targeting domain.

In a further aspect, described herein is a method for delivering a cargo intracellularly, the method comprising providing a composition as defined herein, and administering the composition to target cells in vitro or in vivo.

In a further aspect, described herein is a method for preparing a cargo for intracellular delivery having increased stability, the method comprising covalently linking the cargo to a sufficient number of steroid acid-peptide moieties to produce a covalently-modified cargo that exhibits greater stability (e.g., thermal stability) than the corresponding unmodified cargo.

In a further aspect, described herein is a composition comprising an antigen covalently linked to and/or admixed with a steroid acid-peptide conjugate in an amount sufficient to improve presentation of the antigen upon administration of the composition to non-professional antigen presenting cells (e.g., mesenchymal stromal cells [MSCs]), as compared to administration of a corresponding composition lacking the steroid acid-peptide conjugate.

In a further aspect, described herein is a cell culture comprising non-antigen presenting cells pulsed with an antigen covalently linked to and/or admixed with a steroid acid-peptide conjugate.

In a further aspect, described herein is a vaccine comprising a composition as described herein, or comprising cells produced using a cell culture as described herein.

In a further aspect, described herein is a method for enhancing presentation of an antigen of interest in a subject or cells, the method comprising administering to the subject or in non-antigen presenting cells a composition as described herein, or cells produced using a cell culture as described herein.

In a further aspect, described herein is a method for vaccinating a subject against an infectious disease, the method comprising administering to the subject a composition as described herein or cells produced using a cell culture as described herein, wherein the antigen comprises an antigenic fragment of a pathogen (e.g., virus, bacteria, fungus) causing the infectious disease.

In a further aspect, described herein is a method for treating cancer in a subject, the method comprising administering to the subject a composition as described herein, or cells produced using a cell culture as described herein, wherein the antigen is an overexpressed or aberrantly expressed in cells causing the cancer.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one,” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows representative fluorescence microscopy images for HEK293 cells incubated for 3 or 6 hours with a Cas9-NLS cargo, followed by fixation, permeabilization and labeling with Hoescht nuclear stain (“nuclei”) and a fluorescent anti-Cas9 antibody (“Cas9-NLS cargo”).

FIG. 2 shows representative fluorescence microscopy images for HEK293 cells incubated for 3 or 6 hours with a [CDCA-SV40]-Cas9-NLS cargo, followed by fixation, permeabilization and labeling with Hoescht nuclear stain (“nuclei”) and a fluorescent anti-Cas9 antibody (“[CDCA-SV40]-Cas9-NLS cargo”).

FIG. 3 shows a representative experiment of GFP-Gal3-expressing DC2.4 cells treated with OVA cargo for 3 hours, followed by visualization by fluorescence microscopy. The diffuse cytosolic pattern of GFP-Gal3 is indicative of intact endosomal membranes.

FIG. 4 shows a representative experiment of GFP-Gal3-expressing DC2.4 cells treated with [CA-SV40]-OVA cargo for 3 hours, followed by visualization by fluorescence microscopy. The punctate pattern of GFP-Gal3 is indicative of disrupted endosomal membranes (arrows).

FIG. 5 shows the results of a representative flow-cytometry experiment investigating the intracellular degradation and processing in primary dendritic cells of DQ™ OVA cargo (grey peak) versus [CA-SV40]-DQ™ OVA cargo (red peak) after 3 or 6 hours via flow cytometry.

FIG. 6 shows increased intracellular delivery of OVA-AF647 cargo in mesenchymal stromal cells upon coincubation with CA-hnRPA1 M9 NLS conjugate as evaluated by flow cytometry.

FIG. 7 shows increased intracellular degradation/processing in mesenchymal stromal cells of DQ™ OVA cargo upon coincubation with CA-hnRPA1 M9 NLS conjugate as evaluated by flow cytometry.

FIGS. 8A and 8B show schematic representations of the study design in which Cytochrome C cargo is delivered intracellularly in the absence (FIG. 8A) or presence (FIG. 8B) of CA-SV40 NLS conjugate. FIG. 8C shows a representative flow cytometry assessment of EL4 cell death when treated with CA-SV40 (47 μM) admixed with Cytochrome C.

FIG. 9 shows an intrinsic tryptophan fluorescence (ITF) analysis of unconjugated OVA (“nOVA”) or [CA-SV40]-OVA (“cOVA”) at various CA-SV40 to OVA ratios in response to thermal stress.

FIG. 10A shows a representative gel image of Genomic Cleavage Detection Assay using Control Template & Primers and stained with ethidium bromide. After re-annealing, samples were treated with and without Detection Enzyme and separated on a 2% agarose gel. FIG. 10B shows the cleavage efficiency calculated by determining the relative proportion of DNA contained in each band (parental and cleaved bands) using desired gel analysis software and following the next equations: Cleavage Efficiency=1-[(1-fraction cleaved) 2]; Fraction Cleaved=sum of cleaved band intensities/(sum of the cleaved and parental band intensities).

FIG. 11 shows the effect of different bile acids on the intracellular delivery and subsequent antigen presentation activity of bile acid-SV40 NLS conjugates. For this experiment, BMDCs were used as the antigen presenting cells (n=6) and the molar ratio (bile acid/peptide/conjugate): antigen was 4:1. Controls tested included no antigen (“PBS”), antigen alone (“OVA alone”), unconjugated NLS peptide (“SV40NLS”), unconjugated cholic acid mixed with OVA (“CA”), and the positive control peptide SIINFEKL (SEQ ID NO: 16) mixed with OVA (“SIINFEKL”). The dashed line represents the signal obtained with the OVA cargo alone. Bile acids: cholic acid (CA); glycodeoxycholic acid (GDCA); glycochenodeoxycholic acid (GCDCA); ursodeoxycholic acid (UDCA); and lithocholic acid (LCA).

FIG. 12 shows the effect of different NLS peptides on the intracellular delivery and subsequent antigen presentation activity of cholic acid-NLS peptide conjugates. The dashed line represents the signal obtained with the OVA cargo alone. The readout was taken after 24 h of incubation and error bars represent SD (n=6). For this experiment, BMDCs were used as the antigen presenting cells.

FIG. 13 shows the effect of different NLS peptides on the intracellular delivery and subsequent antigen presentation activity of cholic acid-NLS peptide conjugates. The dashed line represents the signal obtained with the OVA cargo alone. Readout was taken after 24 h of incubation from a single experiment. The molar ratio of CA-peptide conjugate: OVA was 22:1. For this experiment, BMDCs were used as the antigen presenting cells.

FIG. 14 shows the effect of different NLS peptides on the intracellular delivery and subsequent antigen presentation activity of cholic acid-NLS peptide conjugates. For this experiment, BMDCs were used as the antigen presenting cells. The dashed line represents the signal obtained with the OVA cargo alone. Readout was taken after 24 h of incubation from a single experiment. The molar ratio of CA-peptide conjugate: OVA was as follows: CA-GWG-SV40NLS (12:1); CA-hnRNP M NLS (12:1); CA-NLS2-RPS17 NLS (22:1); CA-HuRNLS (22:1); CA-cMyc NLS (2:1); CA-NLS3-RPS17 NLS (22:1); CA-NLS2-RG-RPS17 NLS (2:1); CA-PQBP1 NLS (8:1); CA-hnRNPA1 M9 NLS (22:1); and CA-SV40 NLS (2:1).

FIG. 15 shows the effect of different NLS peptides on the intracellular delivery and subsequent antigen presentation activity of cholic acid-NLS peptide conjugates. For this experiment, a cross-presentation mesenchymal stromal cell (MSC) line was used as the antigen presenting cells. The dashed line represents the signal obtained with the OVA cargo alone. Readout was taken after 24 h of incubation. The molar ratio of CA-peptide conjugate: OVA was as follows: CA-GWG-SV40NLS (2:1); CA-hnRNP M NLS (8:1); CA-hnRNP D NLS (12:1); CA-NLS2-RG-RPS17 (4:1); CA-cMyc NLS (12:1); CA-HuR NLS (12:1); CA-Tus NLS (2:1); CA-NLS2-RPS17 NLS (4:1); CA-PQBP1 NLS (12:1); CA-hnRNPA1 M9 NLS (2:1); and CA-SV40 NLS (2:1).

FIG. 16 shows the effect of different NLS peptides on the intracellular delivery of cholic acid-NLS peptide conjugates. For this experiment, a cross-presenting mesenchymal stromal cell line (cpMSC) was used as the antigen presenting cells, which were pulsed with the OVA cargo labelled with Alexa Fluor 647 (i.e., OVA⁶⁴⁷)^{TM}. OVA⁶⁴⁷ fluorescence was measured by flow cytometry. Different ratios of CA (NLS1 RPS17 [FIG. 16A]; NLS3 RPS17 [FIG. 16B]; and PQBP-1 [FIG. 16C] to antigen (CA: OVA=22:1, 12:1, 8:1, 4:1, and 2:1) were tested (hnRNPA1 M9 NLS at 2:1).

FIG. 17 shows the effect of different NLS peptides on intracellular delivery and subsequent antigen processing activity of cholic acid-NLS peptide conjugates. For this experiment, a cross-presenting mesenchymal stromal cell line (cpMSC) was used as the antigen presenting cells, which were pulsed with DQ™ Ovalbumin (i.e., OVADQ). OVADQ fluorescence was measured by flow cytometry. Different ratios of CA (NLS1 RSP17 [FIG. 17A]; NLS3 RPS17 [FIG. 17B]; and PQBP-1 [FIG. 17C] to antigen (CA: OVA=22:1, 12:1, 8:1, 4:1, and 2:1) were tested (hnRNPA1 M9 NLS at 2:1).

FIG. 18 shows the characterization of the cross-presentation capacity WT MSCs treated with CA-hnRNPA1 (SEQ ID NO: 4). FIG. 18A and FIG. 18B show the results of antigen cross-presentation assay conducted using CA-hnRNPA1 admixed with OVA compared to controls and CA-SV40. FIG. 18C shows the effect of CA-hnRNPA1 on fluorescent OVA uptake by MSCs. FIG. 18D shows the effect of CA-hnRNPA1 on fluorescent OVA processing by MSCs. FIG. 18E shows the results of the antigen cross-presentation assay conducted using different pulsing time points. FIG. 18F shows the results of the antigen cross-presentation assay conducted using CA-hnRNPA1 diluted in PBS or H₂O. FIG. 18G shows a representative flow cytometry analysis of H2-Kb expression. FIG. 18H shows a representative flow-cytometry analysis of I-Ab expression. FIG. 18I shows the phenotype characterization of CA-hnRNPA1-treated MSCs. For FIGS. 18B, 18E and 18F, n=6/group with *P<0.05, **P<0.01 and ***P<0.001.

FIG. 19 shows the antigen cross-presentation capacity WT MSCs treated with CA-hnRNPA1 requires reactive oxygen species (ROS) production. FIG. 19A shows the flow cytometry assessment of ROS production by MSCs in response to CA-hnRNPA1. Dp44mt was used as a positive control. FIG. 19B shows the antigen cross-presentation capacity of CA-hnRNPA1 in the presence of α-tocopherol, MitoTempo™, and N-acetylcysteine (NAC). FIG. 19C shows the antigen cross-presentation capacity of CA-hnRNPA1 can be neutralized using NOX inhibitors diphenyleneiodonium chloride (DPI) and ML171. FIG. 19D shows the endosomal damaging properties of CA-hnRNPA1 on MSCs co-treated with recombinant cytochrome C. For FIGS. 19A-19C, n=6/group with *P<0.05, **P<0.01 and ***P<0.001.

FIG. 20 shows the molecular characterization of the impact of CA-hnRNPA1 on WT MSCs. List of top reactome pathways that are enriched for both up-regulated (FIG. 20A) and down-regulated (FIG. 20B) genes in CA-hnRNPA1 treated group versus control MSCs. Coloured circles intensity corresponds to adjusted p-values; size of circles is the ratio of genes in the tested set. FIG. 20C shows a representative unfolded-protein response heatmap displaying the genes most contributing to the pathway enrichment and modulated in response to CA-hnRNPA1 treatment (FDR <5%); gene expression is scaled between −1 and +1. FIG. 20D shows the bile acid heatmap depicting genes that are modulated by CA-hnRNPA1 treatment. FIG. 20E shows the cholesterol heatmap depicting genes that are modulated by CA-hnRNPA1 treatment. Genes showing in heatmap FIG. 20D and FIG. 20E were also contributing to significant statistics form both differential expression and pathway analyses (FDR <5%). FIG. 20F shows the IL-12 heatmap depicting genes that are modulated by CA-hnRNPA1 treatment. Gene expression is scaled to −1 and 1 range. FIG. 20G shows a Luminex™ analysis of various cytokines in response to CA-hnRNPA1 treatment (in grey). For this figure, n=6/group. FIG. 20H shows the similarity of gene expression patterns between the CA-hnRNPA1 and CA-hnRNPA1+OVA groups compared to control MSCs. Correlation plot showing the spearman's rank correlation coefficient of DEGs (log 2 fold changes). FIG. 20I shows a volcano plot representing differentially expressed genes in response to CA-hnRNPA1. FIG. 20J shows a volcano plot depicting some important biological processes modulated in MSCs in response to CA-hnRNPA1. All genes from corresponding reactome analyses and showing a log 2FC greater or equal to 0.5 are labelled for further investigation. FIG. 20K shows a turbidity assay reflecting the CA-hnRNPA1 capacity to form protein aggregation mixed with the OVA protein.

FIG. 21 shows the validation of the antigen cross-presentation properties of CA-hnRNPA1 on human WT MSCs. FIG. 21A shows the representative flow cytometry analysis of OVA uptake by CA-hnRNPA1-treated human MSCs. FIG. 21B shows the signal quantification of the results presented in FIG. 21A. FIG. 21C shows the representative flow cytometry analysis of OVA processing by CA-hnRNPA1-treated human MSCs. FIG. 21D shows the signal quantification of the results presented in FIG. 21C.

FIG. 22 shows the working model of CA-hnRNPA1-mediated enhancement of antigen cross-presentation in WT MSCs and WT MSC vaccination.

FIG. 23 shows the therapeutic vaccination using the WT MSC vaccine with CA-hnRNPA1 in established lymphoma tumors. FIG. 23A shows the timeline representing the steps used for therapeutic vaccination.
FIG. 23B shows tumor growth in response to syngeneic MSC vaccination. FIG. 23C shows the Kaplan-Meier survival curve of the experiment shown in FIG. 23B. FIG. 23D shows the tumor growth in response to allogeneic MSC vaccination. FIG. 23E shows Kaplan-Meier survival curve of the experiment shown in FIG. 23D. For FIGS. 23B-23E, n=5/group.

### SEQUENCE LISTING

This application contains a Sequence Listing in computer readable form Dec. 8, 2022. The computer readable form is incorporated herein by reference.

| SEQ ID NO: | Description |
|---|---|
| 1 | CA-SV40 |
| 2 | NLS from SV-40 large T-antigen |
| 3 | GWG-SV40NLS |
| 4 | hnRNPA1 M9 NLS |
| 5 | hnRNP D NLS |
| 6 | hnRNP M NLS |
| 7 | PQBP-1 NLS |
| 8 | NLS2-RG Domain RPS17 |
| 9 | NLS1 RPS17 |
| 10 | NLS2 RPS17 |
| 11 | NLS3 RPS17 |
| 12 | cMyc NLS |
| 13 | HUR NLS |
| 14 | Tus NLS |
| 15 | Nucleoplasmin NLS |
| 16 | SIINFEKL peptide |

### DETAILED DESCRIPTION

Described herein are compositions and methods relating to cargoes for improved intracellular delivery. In some aspects, the present invention stems from the demonstration herein that total intracellular delivery, cytosolic delivery, and/or nuclear delivery of cargoes may be enhanced by admixture with, or covalent linkage to, a variety of steroid acid-peptide conjugates. In further aspects, the present invention stems from the demonstration herein that covalent conjugation with steroid acid-peptide moieties may improve cargo stability. In further aspects, the present invention stems from the demonstration herein that steroid acid-peptide conjugates described herein may be used for the generation of cell-based vaccines, including in some non-professional antigen-presenting cells that have been previously shown to be immunosuppressive. In a first aspect, described herein is a composition comprising a steroid acid-peptide conjugate covalently linked to and/or admixed with a cargo for intracellular delivery. In some embodiments, covalently linking the cargo to the steroid acid-peptide conjugate increases intracellular delivery and/or cytosolic/nuclear delivery of the cargo, as compared to a corresponding composition lacking the steroid acid-peptide conjugate. In some embodiments, the cargo is admixed with a sufficient concentration of the steroid acid-peptide conjugate to increase intracellular delivery and/or cytosolic/nuclear delivery of the cargo, as compared to a corresponding composition lacking admixture with the steroid acid-peptide conjugate. In some embodiments, steroid acid-peptide conjugate conjugates described herein may increase presentation of an antigenic polypeptide cargo by target cells. In some embodiments, steroid acid-peptide conjugate conjugates described herein may increase intracellular reactive oxygen species production in target cells. In some embodiments, the target cells comprise or consist of professional anti-presenting cells (e.g., dendritic cells, macrophages, B cells, or non-immune cells engineered for overexpression of an immunoproteasome). In some embodiments, the target cells comprise or consist of non-professional antigen-presenting cells (e.g., wild-type, engineered, primary, and/or cultured non-immune cells, such as mesenchymal stromal cells [MCSs; also known as mesenchymal stem cells]. In some embodiments, steroid acid-peptide conjugate conjugates described herein may transform immunosuppressive cells (e.g., immunosuppressive MSCs) into immunostimulatory and/or proinflammatory MSCs, which may then be used, for example, in cell-based immunostimulatory compositions and/or cell-based vaccines.

In some embodiments, the cargo may be or may comprise a protein, peptide, polynucleotide (e.g., DNA, RNA, shRNA, siRNA, antisense oligonucleotides), polynucleotide analog (having cationic, anionic, or charge-neutral backbones), polysaccharide, drug, or any combination thereof. In some embodiments, the cargo for intracellular delivery is a cargo that does not bind specifically to a cell surface receptor or ligand such that increased intracellular delivery is not predominantly the result of receptor- or ligand-mediated internalization/endocytosis (e.g., as is the case with antibody-drug conjugates). In some embodiments, the cargo is not an antibody (e.g., an antibody that binds to a cell surface epitope). In some embodiments, the cargo may comprise an antibody or fragment thereof that specifically binds to an intracellular target or epitope. In some embodiments, the cargo is not an antigen against which an immune response is to be mounted. In some embodiments, compositions described herein do not comprise an adjuvant and/or are not formulated as immunogenic or vaccine compositions.

In some embodiments, the compositions described herein may be for use in genome editing, base editing, transcription control/regulation, o diagnostic compositions. In some embodiments, cargoes described herein may include RNA- or DNA guided nucleases having or lacking DNA/RNA cleavage activity. In some embodiments, cargoes described herein may comprise a CRISPR-Cas nuclease, such as a class 2 CRISPR-Cas nuclease. In some embodiments, the cargoes described herein may comprise Cas9 or Cas12a.

In some embodiments, the cargo described herein may be covalently linked to a sufficient number of steroid acid-peptide moieties such that the cargo exhibits greater stability (e.g., thermal stability) than the unmodified cargo.

In some embodiments, the steroid acid in the steroid acid-peptide conjugates or moieties described herein may enhance endocytosis and/or endosomal escape when internalized. Without being bound by theory, steroid acids (e.g., bile acids and bile acid analogs) have been shown to be utilized/exploited by viruses to facilitate their infection of host cells, such as by increasing their endocytic uptake and/or endosomal escape to gain access to the cytosol (Shivanna et al., 2014; Shivanna et al., 2015; Murakami et al., 2020). For example, bile acids have been shown to trigger the enzyme acid sphingomyelinase (ASM) to cleave sphingomyelin to ceramide on the inner leaflet of endosomes. Increased amounts of ceramide destabilize membranes and facilitate endosomal escape. In some embodiments, steroid acids described herein may comprise those that trigger ceramide accumulation on the inner leaflet of endosomes, thereby destabilizing endosomal membranes and facilitating endosomal escape of the steroid acid upon intracellular delivery. In some embodiments, steroid acids described herein comprise those that trigger increased acid sphingomyelinase (ASM)-mediated cleavage of sphingomyelin to form ceramide.

In some embodiments, the steroid acid described herein may comprise or consist of a bile acid (e.g., a primary bile acid or a secondary bile acid). In some embodiments, the steroid acid may be or comprise: cholic acid (CA), chenodeoxycholic acid (CDCA), deoxycholic acid (DCA), lithocholic acid (LCA), glycodeoxycholic acid (GDCA), glycocholic acid (GCA), taurocholic acid (TCA), glycodeoxycholic acid (CDCA), glycochenodeoxycholic acid (GCDCA), taurodeoxycholic acid (TDCA), glycolithocholic acid (GLCA), taurolithocholic acid (TLCA), taurohyodeoxycholic acid (THDCA), taurochenodeoxycholic acid (TCDCA), ursocholic acid (UCA), tauroursodeoxycholic acid (TUDCA), ursodeoxycholic acid (UDCA), glycoursodeoxycholic acid (GUDCA), or any analog thereof that: induces endocytosis; triggers ceramide accumulation on the inner leaflet of endosomes; triggers increased acid sphingomyelinase (ASM)-mediated cleavage of sphingomyelin to form ceramide; and/or has a hydrophobicity greater than that of cholic acid.

Hydrophobic bile acids such as GCDCA, TCA, GCA, and CA (but not hydrophilic bile acids such as UDCA) were shown to increase GII.3 human norovirus infection and replication in host intestinal cells by enhancing endosomal uptake and endosomal escape via ASM-mediated ceramide accumulation on the apical membrane (Murakami et al., 2020). In some embodiments, the steroid acid described herein comprises or consists of a bile acid or bile acid analog that is more hydrophobic than cholic acid. In some embodiments, the steroid acid described herein comprises or consists of a bile acid or bile acid analog that is more hydrophobic than cholic acid (e.g., CDCA, DCA, LCA, TCA, TDCA, TCDCA, GCA, GDCA, or GCDCA; Hanafi et al., 2018).

In some embodiments, the cargoes described herein are covalently linked to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 steroid acid-peptide moieties. Covalent modification of proteinaceous antigens with different steroid acid-NLS peptides has been shown in previous studies to enhance intracellular delivery and antigen presentation in a variety of antigen-presenting cells (U.S. Pat. No. 11,291,717).

In some embodiments, the steroid acid-peptide conjugate is covalently linked to the cargo via a linker (e.g., bifunctional, trifunctional linker, or multi-functional linker). In some embodiments, the linker may be a cleavable or non-cleavable linker.

In some embodiments, the steroid acid may be conjugated to the peptide, for example at or towards a free N-terminal or C-terminal amino group of the peptide or at some other functional group within the peptide.

In some embodiments, the peptide may be a non-immunogenic peptide. In some embodiments, the peptide may be a water-soluble peptide, wherein conjugation of the peptide to the steroid acid increases the water solubility of the steroid acid-peptide moiety as compared to the steroid acid moiety alone. In some embodiments, the peptide may be a cationic peptide. In some embodiments, the peptide may comprise one or more domains that impart an additional functionality to the modified polypeptide antigen. As used herein, a “domain” generally refers to a part of a protein having a particular functionality. Some domains conserve their function when separated from the rest of the protein, and thus can be used in a modular fashion. The modular characteristic of many protein domains can provide flexibility in terms oftheir placement within the peptides described herein. However, some domains may perform better when engineered at certain positions of the peptide (e.g., at the N- or C-terminal region, or therebetween). The position of the domain within its endogenous protein may be an indicator of where the domain should be engineered within the peptide.

In some embodiments where non-specific delivery may be desired, the peptide may comprise a protein transduction domain (PTD) that stimulates endocytosis, endosomal formation, or intracellular delivery in a non-cell-specific manner. In some embodiments, the peptide may comprise a subcellular targeting signal promoting targeting of the modified polypeptide antigen to a specific subcellular compartment. In some embodiments, the peptide may comprise a nuclear localization signal (NLS) that targets the modified polypeptide antigen to the nucleus.

In some embodiments, the nuclear localization signals described herein may comprise or be derived from the NLS from SV-40 large T-antigen (e.g., PKKKRKV; SEQ ID NO: 1 or 2) or from other classical NLSs. In some embodiments, the nuclear localization signals described herein may comprise or be derived from non-classical NLS (e.g., acidic M9 domain in the hnRNP A1 protein; the sequence KIPIK in yeast transcription repressor Matα2; PY-NLS; ribosomal NLS; or the complex signals of U snRNPs). In some embodiments, the nuclear localization signal described herein comprises or consists essentially of the amino acid sequence of any one of SEQ ID NOs: 1 to 15, or any portion thereof. In some embodiments, the nuclear localization signal described herein comprises or consists essentially of a nuclear localisation signal which is SV40 NLS (e.g., comprised in SEQ ID NO: 1 or 2), GWG-SV40 NLS (e.g., comprised in SEQ ID NO: 3), hnRNPA1 M9 NLS (e.g., comprised in SEQ ID NO: 4), hnRNP D NLS (e.g., comprised in SEQ ID NO: 5), hnRNP M NLS (e.g., comprised in SEQ ID NO: 6), PQBP-1 NLS (e.g., comprised in SEQ ID NO: 7), NLS2-RG Domain RPS17 (e.g., comprised in SEQ ID NO: 8), NLS1 RPS17 (e.g., comprised in SEQ ID NO: 9), NLS2 RPS17 (e.g., comprised in SEQ ID NO: 10), NLS3 RPS17 (e.g., comprised in SEQ ID NO: 11), cMyc NLS (e.g., comprised in SEQ ID NO: 12), HuR NLS (e.g., comprised in SEQ ID NO: 13), Tus NLS (e.g., comprised in SEQ ID NO: 14), or Nucleoplasmin NLS (e.g., comprised in SEQ ID NO: 15). In some instances, the SEQ ID NOs referred to above comprise an N-terminal cysteine residue that was or that may be used to facilitate conjugation to the polypeptide antigen (e.g., the thiol group of the N-terminal cysteine residue). Thus, in some embodiments, the NLS sequences referred to herein may exclude the N-terminal cysteine residue comprised in any one of SEQ ID NOs: 1 to 15. In some embodiments, other functional groups added or inserted (e.g., towards the N or C terminal portions of the peptides described herein) to facilitate steroid acid-peptide conjugation to a given polypeptide antigen are also envisaged (e.g., carboxyl groups, synthetic amino acids, etc.). For example, the peptide may include a C-term amide and/or an N-term cysteine.

In some embodiments, the peptide describe herein may comprise one or more cysteine residues (e.g., at or towards the peptide's N- and/or C terminus) having a free thiol group (—SH) or a thiol group that is protected in a cleavable manner (e.g., by a pharmaceutically acceptable protecting group). Such modifications may be introduced, for example, during chemical synthesis of the peptides or via chemical modification with one or more functional or protecting groups following peptide synthesis. In some embodiments, free thiol groups facilitate further conjugations and/or reactivities in reducing environments, such as the peri-cellular and/or intracellular environments (e.g., of cancer cells). In some embodiments, the free thiol group may be conjugated or protected by conjugation to a different peptide or to the same peptide (e.g., via a disulphide bridge between two cysteine-comprising peptides). In some embodiments, the steroid acid-peptide conjugates described herein may be comprised in oligomer form (e.g., dimer, trimer, tetramer, pentamer, etc.). In some embodiments, the steroid acid-peptide conjugates described herein may be comprised in oligomer form via cleavable linkages (e.g., disulphide or other linkages cleavable in peri-cellular and/or intracellular environments). In some embodiments, the cleavable linkages maybe motifs recognizable by intracellular proteases.

In some embodiments, peptides described herein do not comprise an endosomal escape motif, or protein transduction, or cell penetrating motif.

In some embodiments, the nuclear localization signals described herein may comprise the general consensus sequence: (i) K(K/R)X(K/R); (ii) (K/R)(K/R)X₁₀₋₁₂(K/R)_{3/5}, wherein (K/R)_{3/5} represents three lysine or arginine residues out of five consecutive amino acids; (iii) KRX₁₀₋₁₂KRRK; (iv) KRX₁₀₋₁₂K(K/R)(K/R); or (v) KRX₁₀₋₁₂K(K/R)X(K/R), wherein X is any amino acid (Sun et al., 2016).

In some embodiments, the peptide does not include an endosomal escape motif (e.g. -GFFG, -GWG, -GFWG, -GFWFG, -GWWG, -GWGGWG, and -GWWWG), or protein transduction, or cell penetrating motif (such as a cell penetrating peptide).

In some embodiments, the steroid acid described herein is not or does not comprise cholic acid; the NLS peptide is not or does not comprise an SV40 NLS; and/or the steroid acid-peptide conjugate is not or does not comprise CA-SV40.

In some embodiments, the composition may further comprise any pharmaceutically or physiologically acceptable carrier and/or excipient. In some embodiments, the compositions described herein may be formulated within a hydrogel, liposome, lipid-based transfection agent, or nanoparticle (e.g., lipid nanoparticle).

In some embodiments, the compositions, methods and uses described herein may be formulated or adapted for any route of administration, such as but not limited to oral, intravenous, intranasal, intramuscular, subcutaneous, intradermal, intratumoral, intracranial, topical, and intrarectal administration.

In some embodiments, the compositions described herein may be for use in increasing the intracellular, cytosolic, and/or nuclear delivery of a biologically active cargo (e.g., therapeutic cargo or diagnostic cargo) in vitro or in vivo.

In a further aspect, described herein is a method for delivering a cargo intracellularly, the method comprising providing a composition as defined herein, and administering the composition to target cells in vitro or in vivo.

In a further aspect, described herein is a method for preparing a cargo for intracellular delivery having increased the stability, the method comprising covalently linking the cargo to a sufficient number of steroid acid-peptide moieties to produce a covalently-modified cargo that exhibits greater stability (e.g., thermal stability) than the corresponding unmodified cargo. In some embodiments, cargo may be reacted with between a 2-fold and 100-fold molar excess of the steroid acid-peptide conjugate; between a 2-fold and 50-fold molar excess of the steroid acid-peptide conjugate; or between a 5-fold and 25-fold molar excess of the steroid acid-peptide conjugate. In some embodiments, the mean number of steroid acid-peptide moieties conjugated per cargo is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50; or is between about 1, 2, 3, 4, 5, 6, 7, 8, 9, or 10 and n, wherein n is the total number of accessible sites on the cargo available for conjugation.

In another aspect, described herein is a composition comprising non-antigen presenting cells or non-professional antigen presenting cells and an antigen covalently linked to or admixed with an enhancer of antigen-presentation (e.g., the steroid acid-peptide conjugate defined herein). As used herein, the term “admixture” or “admixing” refers to the combination of two separate components into a single composition, wherein the components are not covalently conjugated or otherwise reacted together. In some embodiments, the enhancer may comprise a steroid acid or steroid acid-peptide conjugate in an amount sufficient to improve presentation (e.g., cross presentation or classical antigen presentation) of the antigen upon administration of the composition to non-antigen-presenting cells (e.g., in vitro, ex vivo, or in vivo), as compared to administration of a corresponding composition lacking the enhancer. In some embodiments, the enhancer may comprise a steroid acid-peptide conjugate in an amount sufficient to improve presentation (e.g., cross presentation or classical antigen presentation) of the antigen upon administration of the composition to antigen-presenting cells (e.g., in vitro, ex vivo, or in vivo), as compared to administration of a corresponding composition lacking the enhancer.

As used herein, the term “non-antigen presenting cells (APCs)” or “non-professional antigen presenting cells” refer to cells that do not efficiently present antigen or possess the ability/machinery to efficiently present antigen when unstimulated, untreated, or unmodified (e.g., genetically). For example, untreated wild type mesenchymal stem cells do not efficiently present antigen to T cells, whereas they can be genetically engineered to possess specific machinery (e.g., proteasomes or immunoproteosomes) to enhance their antigen presentation capabilities. Professional APCs generally refer to dendritic cells (DCs), macrophages, and B cells, which express high levels of MHC-II and express sufficient levels of the proteins/machinery involved in efficient antigen presentation.

As used herein, the term “antigen presentation” may refer to the classical antigen presentation pathways of extracellular (via MHC class II) and/or intracellular antigens (via MHC class I), as well as the cross presentation pathway (presentation of extracellular antigen via MHC class I).

Polypeptide antigens are normally captured by antigen-presenting cells (e.g., dendritic cells) but are initially entrapped in endosomes. Endosomal maturation towards lysosomes results in a decrease in pH and an activation of proteolytic enzymes that mediate non-specific antigen degradation. As a result, some of the antigen fragments generated may then pass through endosomal pores to reach the cytosol where further antigen degradation takes place by the proteasomal machinery prior to MHC class I presentation. Although this process occurs naturally, the generated antigen fragments that ultimately leave the endosomes may be small and/or damaged, rendering them unsuitable for proteasomal degradation, thereby precluding their MHC class I presentation and thus cellular immunity based thereon. Without being bound by theory, admixture of antigens with immunogen enhancers described herein may facilitate internalization/endosomal escape of the antigens, allowing them (or larger antigen fragments) to reach the cytosol in a more native conformation and/or in greater quantities. As a result, proteasomal degradation of these more native antigens may result in a higher amount and/or variety of immunogenic and/or stable peptides presented via MHC class I at the surface of antigen-presenting cells, thereby eliciting potent T-cell activation.

As used herein, “polypeptide antigen” refers to an immunogenic peptide-linked chain of amino acids of any length, but generally at least 8, 9, 10, 11, or 12 amino acids long. For greater clarity, polypeptide antigens referred to herein exclude antigen-binding antibodies or fragments thereof. As used herein, a “protein antigen” refers to a polypeptide antigen having a length of at least 50 amino acid residues, while a “peptide antigen” refers to a polypeptide antigen having a length of less than 50 amino acid residues. For greater clarity, polypeptides, proteins, and peptides described herein may or may not comprise any type of modification (e.g., chemical or post-translational modifications such as acetylation, phosphorylation, glycosylation, sulfatation, sumoylation, prenylation, ubiquitination, etc.) or incorporate one or more synthetic or non-natural amino acids, to the extent that the modification or synthetic or non-natural amino acids does not destroy the antigenicity of the polypeptide antigen or the desired functionality of the peptide (or domain comprised therein).

### ITEMS

1. A composition comprising a steroid acid-peptide conjugate covalently linked to and/or admixed with a cargo for intracellular delivery.

2. The composition of item 1, wherein the cargo is or comprises a protein, peptide, polynucleotide, polynucleotide analog, polysaccharide, drug, or any combination thereof.

3. The composition of item 1 or 2, wherein: (a) the cargo does not bind specifically to a cell surface receptor or ligand; (b) the cargo is not an antibody (e.g., an antibody that binds to a cell surface epitope); (c) the cargo is not or does not comprise an antigen; or (d) any combination of (a) to (c).

4. The composition of any one of items 1 to 3, wherein the cargo is or comprises a nuclease, such as a CRISPR-Cas nuclease (e.g., a class 2 CRISPR-Cas nuclease, such as Cas9 or Cas12a).

5. The composition of any one of items 1 to 4, wherein: (a) covalently linking the cargo to the steroid acid-peptide conjugate increases intracellular delivery and/or cytosolic/nuclear delivery of the cargo, as compared to a corresponding composition lacking the steroid acid-peptide conjugate; or (b) the cargo is admixed with a sufficient concentration of the steroid acid-peptide conjugate to increase intracellular delivery and/or cytosolic/nuclear delivery of the cargo, as compared to a corresponding composition lacking admixture with the steroid acid-peptide conjugate.

6. The composition of any one of items 1 to 5, wherein the cargo is covalently linked to a sufficient number of steroid acid-peptide moieties such that the cargo exhibits greater stability (e.g., thermal stability) than the unmodified cargo.

7. The composition of any one of items 1 to 6, wherein the steroid acid is or comprises a bile acid (e.g., a primary bile acid or a secondary bile acid).

8. The composition of any one of items 1 to 7, wherein the steroid acid is or comprises: (a) a bile acid which is: cholic acid (CA), chenodeoxycholic acid (CDCA), deoxycholic acid (DCA), lithocholic acid (LCA), glycodeoxycholic acid (GDCA), glycocholic acid (GCA), taurocholic acid (TCA), glycodeoxycholic acid (CDCA), glycochenodeoxycholic acid (GCDCA), taurodeoxycholic acid (TDCA), glycolithocholic acid (GLCA), taurolithocholic acid (TLCA), taurohyodeoxycholic acid (THDCA), taurochenodeoxycholic acid (TCDCA), ursocholic acid (UCA), tauroursodeoxycholic acid (TUDCA), ursodeoxycholic acid (UDCA), or glycoursodeoxycholic acid (GUDCA); (b) an analog of the bile acid of (a) that: induces endocytosis; triggers ceramide accumulation on the inner leaflet of endosomes; triggers increased acid sphingomyelinase (ASM)-mediated cleavage of sphingomyelin to form ceramide; and/or has a hydrophobicity greater than that of cholic acid; (c) a bile acid or bile acid analog that is more hydrophobic than cholic acid (e.g. CDCA, DCA, LCA, TCA, TDCA, TCDCA, GCA, GDCA, or GCDCA); or (d) any combination of (a) to (c).

9. The composition of any one of items 1 to 8, wherein each cargo molecule is covalently linked to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 steroid acid-peptide moieties.

10. The composition of any one of items 1 to 9, wherein the steroid acid-peptide conjugate is covalently linked to the cargo via a cleavable or non-cleavable linker (e.g., bifunctional, trifunctional linker, or multi-functional linker).

11. The composition of any one of items 1 to 10, wherein the steroid acid-peptide conjugate is covalently linked to the cargo via said peptide (e.g., the steroid acid is conjugated at or towards the N- or C-terminus of the peptide).

12. The composition of any one of items 1 to 11, wherein the peptide: (i) comprises a protein transduction domain that stimulates endocytosis and/or endosomal formation; (ii) comprises a subcellular targeting signal; (iii) is a cationic peptide (e.g., a non-cell-penetrating cationic peptide); (iv) is a non-immunogenic peptide; (v) comprises at least one cysteine residue (e.g., at or towards the peptide's N- and/or C terminus) having a free thiol group or a thiol group that is protected in a cleavable manner (e.g., by a pharmaceutically acceptable protecting group); or (vi) any combination of (i) to (v).

13. The composition of any one of items 1 to 12, wherein: the steroid acid is not or does not comprise cholic acid; the NLS peptide is not or does not comprise an SV40 NLS; and/or the steroid acid-peptide conjugate is not or does not comprise CA-SV40.

14. The composition of any one of items 1 to 13, wherein the peptide is or comprises a nuclear localization signal which is a classical NLS (e.g., NLS from SV-40 large T-antigen (e.g., PKKKRKV; SEQ ID NO: 1 or 2) or from other classical NLSs) or a non-classical NLS (e.g., acidic M9 domain in the hnRNP A1 protein; the sequence KIPIK in yeast transcription repressor Matα2; PY-NLS; ribosomal NLS; and the complex signals of U snRNPs).

15. The composition of any one of item 1 to 14, wherein the peptide is or comprises a nuclear localization signal which is a/an: SV40 NLS (e.g., comprised in SEQ ID NO: 1 or 2), GWG-SV40NLS (e.g., comprised in SEQ ID NO: 3), hnRNPA1 M9 NLS (e.g., comprised in SEQ ID NO: 4), hnRNP D NLS (e.g., comprised in SEQ ID NO: 5), hnRNP M NLS (e.g., comprised in SEQ ID NO: 6), PQBP-1 NLS (e.g., comprised in SEQ ID NO: 7), NLS2-RG Domain RPS17 (e.g., comprised in SEQ ID NO: 8), NLS1 RPS17 (e.g., comprised in SEQ ID NO: 9), NLS2 RPS17 (e.g., comprised in SEQ ID NO: 10), NLS3 RPS17 (e.g., comprised in SEQ ID NO: 11), cMyc NLS (e.g., comprised in SEQ ID NO: 12), HuR NLS (e.g., comprised in SEQ ID NO: 13), Tus NLS (e.g., comprised in SEQ ID NO: 14), or Nucleoplasmin NLS (e.g., comprised in SEQ ID NO: 15), or is a variant of an NLS having nuclear localization activity, the NLS comprising or consisting of the amino acid sequence of any one of SEQ ID NOs: 1 to 15.

16. The composition of any one of items 1 to 15, wherein the steroid acid comprises CA or DCA, and the peptide comprises an hnRNPA1 M9 NLS or a variant thereof having nuclear localization activity.

17. The composition of any one of item 1 to 16, wherein the peptide does not comprise an endosomal escape motif, or protein transduction motif, or cell penetrating motif.

18. The composition of any one of items 1 to 17, wherein the composition or conjugate is formulated within a hydrogel, liposome, lipid-based transfection agent, or nanoparticle (e.g., lipid nanoparticle).

19. The composition of any one of items 1 to 18, further comprising pharmaceutically or physiologically acceptable carrier and/or excipient.

20. The composition of any one of items 1 to 19, for use in: (a) increasing the intracellular, cytosolic, and/or nuclear delivery of a biologically active cargo (e.g., therapeutic cargo or diagnostic cargo) in vitro or in vivo, as compared to a corresponding composition lacking the steroid acid-peptide conjugate; (b) increasing presentation of an antigenic polypeptide cargo by target cells, such as by professional anti-presenting cells (e.g., dendritic cells, macrophages, B cells, or non-immune cells engineered for overexpression of an immunoproteasome), or by non-professional antigen-presenting cells (e.g., wild-type, engineered, primary, and/or cultured non-immune cells, such as mesenchymal stromal cells (MCSs)); (c) increasing intracellular reactive oxygen species production in target cells, such as by professional anti-presenting cells (e.g., dendritic cells, macrophages, B cells, or non-immune cells engineered for overexpression of an immunoproteasome), or by non-professional antigen-presenting cells (e.g., wild-type, engineered, primary, and/or cultured non-immune cells, such as MCSs); (d) transforming immunosuppressive cells (e.g., immunosuppressive MSCs) into immunostimulatory and/or proinflammatory MSCs; or (e) any combination of (a) to (d).

21. The composition for use of item 20, wherein the composition is adapted or formulated for oral, intravenous, intranasal, intramuscular, subcutaneous, intradermal, intratumoral, intracranial, topical, intrarectal administration, or any other route of administration.

22. A method for delivering a cargo intracellularly, the method comprising providing a composition as defined in any one of items 1 to 21, and administering the composition to target cells in vitro or in vivo.

23. A method for preparing a cargo for intracellular delivery having increased stability, the method comprising covalently linking the cargo to a sufficient number of steroid acid-peptide moieties to produce a covalently-modified cargo that exhibits greater stability (e.g., thermal stability) than the corresponding unmodified cargo.

24. The method of item 23, wherein the cargo and/or the steroid acid-peptide is as defined in any one of items 1 to 17.

25. The method of item 23 or 24, wherein the cargo is reacted or admixed with between a 2-fold and 1000-fold, 2-fold and 500-fold, 2-fold and 200-fold, 2-fold and 100-fold molar excess of the steroid acid-peptide conjugate; between a 2-fold and 50-fold molar excess of the steroid acid-peptide conjugate; or between a 5-fold and 25-fold molar excess of the steroid acid-peptide conjugate.

26. The method of any one of items 23 to 25, wherein the mean number of steroid acid-peptide moieties conjugated per cargo, or the molar ratio of cargo: steroid acid-peptide conjugate admixed, is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50; or wherein the mean number of steroid acid-peptide moieties conjugated per cargo is between about 1, 2, 3, 4, 5, 6, 7, 8, 9, or 10 and n, wherein n is the total number of accessible sites on the cargo available for conjugation.

27. A composition comprising an antigen covalently linked to and/or admixed with a steroid acid-peptide conjugate in an amount sufficient to improve presentation of the antigen upon administration of the composition to non-antigen presenting cells (e.g., mesenchymal stromal cells [MSCs]), as compared to administration of a corresponding composition lacking the steroid acid-peptide conjugate.

28. The composition of item 27, wherein the steroid acid or peptide is as defined in any one of items 7, 8, or 10 to 17.

29. The composition of item 27 or 28, wherein the molar ratio of steroid acid-peptide conjugate to antigen in the composition is at least 0.01:1, 0.05:1, 0.1:1, 0.2:1, 0.5:1, 1:1, 2:1, 3:1, 4:1, 5:1, 6:1, 7:1, 8:1, 9:1, 10:1, 15:1, 20:1; is no more than 2:1, 3:1, 4:1, 5:1, 6:1, 7:1, 8:1, 9:1, 10:1, 15:1, 20:1, 50:1, 100:1, 250:1, 500:1, 1000:1, and/or is between 1:1 to 1000:1; 1:1 to 500:1, 1:1 to 250:1, 1:1 to 200:1.

30. The composition of any one of items 27 to 29, wherein the steroid acid is conjugated to the peptide: (a) at a molar ratio of steroid acid: peptide of 1:1, 2:1, 3:1, 4:1, 5:1, 6:1, 7:1, 8:1, 9:1, or 10:1, or between 1:1 to 10:1; (b) at a free amino group and/or a free thiol group (e.g., of a lysine or cysteine) of the peptide; (c) at or towards the N-terminal end of the peptide (e.g., at the free amino group of N terminal residue and/or at the thiol group of an N-terminal cysteine residue); or (d) any combination of (a) to (c).

31. The composition of any one of items 27 to 30, wherein the antigen is a polypeptide antigen comprising one or more MHC class I epitopes and/or MHC class II epitopes.

32. The composition of any one of items 27 to 31, wherein the antigen is or comprises: (a) a tumor-associated antigen (TAA), tumor-specific antigen (TSA), tumor lysate, a neoantigen, a viral antigen, a bacterial antigen, a fungal antigen, an antigen associated with a disease or disorder amenable to treatment by vaccination and/or immunotherapy; or any antigenic fragment thereof; or (b) a corona viral antigen (e.g., SARS-CoV-2 Spike protein, SARS-CoV Spike protein, or an antigenic fragment thereof; or a cancer antigen, such as a single-nucleotide variant antigen, a mutational frameshift antigen, splice variant antigen, a gene fusion antigen, an endogenous retroelement antigen, or another class of antigen, such as a human leukocyte antigen (HLA)-somatic mutation-derived antigen or a post-translational TSA, a viral-derived cancer antigen (e.g., from human papillomavirus (HPV), cytomegalovirus, or Epstein-Barr virus (EBV)), a cancer-testis antigen, HER2, PSA, TRP-1, TRP-2, EpCAM, GPC3, CEA, MUC1, MAGE-A1, NY-ESO-1, SSX-2, mesothelin (MSLN), EGFR, cell lysates or other material derived from a tumor (e.g., tumor-derived exosomes).

33. The composition of any one of items 27 to 32, further comprising a pharmaceutically acceptable excipient and/or adjuvant.

34. A cell culture comprising non-antigen presenting cells (e.g., mesenchymal stromal cells [MSCs]) and the composition as defined in any one of items 27 to 33.

35. A cell culture comprising non-antigen presenting cells (e.g., mesenchymal stromal cells [MSCs]) pulsed with an antigen covalently linked to and/or admixed with a steroid acid-peptide conjugate.

36. A vaccine comprising the composition as defined in any one of items 27 to 32, or comprising cells produced using the cell culture as defined in item 34 or 35.

37. The vaccine of item 36, which is a therapeutic or prophylactic vaccine (e.g., anti-cancer vaccine, anti-viral vaccine, or anti-bacterial vaccine).

38. A method for enhancing presentation of an antigen of interest in a subject or cells, the method comprising administering to the subject or in non-antigen presenting cells (e.g., mesenchymal stromal cells [MSCs]) the composition as defined in any one of items 27 to 33, or cells produced using the cell culture as defined in item 34 or 35.

39. A method for vaccinating a subject against an infectious disease, the method comprising administering to the subject the composition as defined in any one of items 27 to 33 or cells produced using the cell culture as defined in item 34 or 35, wherein the antigen comprises an antigenic fragment of a pathogen (e.g., virus, bacteria, fungus) causing the infectious disease.

40. A method for treating cancer in a subject, the method comprising administering to the subject the composition as defined in any one of items 27 to 33 or cells produced using the cell culture as defined in item 34 or 35, wherein the antigen is an overexpressed or aberrantly expressed in cells causing the cancer.

41. The composition as defined in any one of items 27 to 33, or the cell culture as defined in item 34 or 35, for use in: (i) generating enhancing presentation of an antigen of interest in a subject or in non-professional antigen presenting cells (e.g., mesenchymal stromal cells [MSCs]); (ii) the manufacture of a medicament (e.g., vaccine) for generating an immune response in a subject; (iii) increasing presentation of an antigenic polypeptide cargo by non-professional antigen-presenting cells (e.g., wild-type, engineered, primary, and/or cultured non-immune cells, such as mesenchymal stromal cells [MCSs]); (iv) increasing intracellular reactive oxygen species production in by non-professional antigen-presenting cells (e.g., wild-type, engineered, primary, and/or cultured non-immune cells, such as MCSs); (v) transforming immunosuppressive cells (e.g., immunosuppressive MSCs) into immunostimulatory and/or proinflammatory MSCs; or (vi) any combination of (i) to (v).

### Examples

### Example 1: General Materials and Methods

### Cell Lines and Reagents

All cell culture media and reagents were purchased from Wisent Bioproducts (St-Bruno, QC, Canada) unless otherwise indicated. All flow cytometry antibodies were purchased from BD Biosciences (San Jose, CA, USA) unless otherwise indicated. The albumin from chicken egg white (ovalbumin; OVA) and LPS was purchased from Sigma-Aldrich (St-Louis, MI, USA). DQ™ OVA was purchased from ThermoFisher (Waltham, MA, USA). Recombinant GM-CSF was purchased from Peprotech (Rocky Hill, NJ, USA).

### Cargo Delivery Fluorescence Microscopy Assay

Sterilized microscopy coverslips were placed in 24-well cell culture plates and seeded overnight with 25,000 cells per well. The following day, cells were treated with a cargo solution (e.g., containing unconjugated cargo; cargo conjugated to one or more steroid acid-peptide moieties; or unconjugated cargo mixed with steroid acid-peptide moieties) in a final volume of 250 μL per well for a specified incubation time. Following incubation, cells were washed three times with PBS and then fixed for 30 minutes in a 1% paraformaldehyde/sucrose solution on ice. The fixed cells were permeabilized with 0.05% Triton X-100/PBS, washed three times with PBS, and then blocked with a 10% normal goat serum/PBS solution for 1 h in a humidified chamber. For Cas9 cargoes, cells were then treated with an anti-Cas9-AF488 antibody and incubated at room temperature in the dark for 1 h, washed three times in PBS, and then incubated with Hoescht nuclear stain diluted in PBS for 15 minutes. After a final washing step, the cells were mounted on microscopy slides with a drop of SlowFade™ reagent and sealed.

### Generation of Bone Marrow Derived DCs

Mouse bone marrow derived DCs (BMDCs) were generated by flushing the whole marrow from mouse femurs using RPMI™ 1640 supplemented with 10% fetal bovine serum (FBS), 50 U/mL Penicillin-Streptomycin, 2 mM L-glutamine, 10 mM HEPES, 1% MEM Non-essential Amino Acids, 1 mM Sodium Pyruvate, 0.5 mM beta-mercaptoethanol. Following red blood cell lysis, cells were then cultured in media supplemented with 50 ng/mL murine recombinant GM-CSF. The media was changed on days 2, 4, 6 and 8. On day 9, the media was replaced to include recombinant murine GM-CSF and LPS from Escherichia coli 0111 (1 ng/mL) to stimulate DC maturation. Mature DCs were assessed by flow cytometry for their surface expression of CD3, CD19, NK1.1, CD11c, CD80, CD86, and I-A^{b}.

### Phenotypic Assessment of Generated BMDCs by Flow Cytometry

To assess the expression of cell surface markers, BMDCs were incubated with various antibodies diluted according to manufacturer's instructions using the staining buffer (PBS containing 2% FBS) for 30 min at 4° C. in the dark. After extensive washing using the staining buffer, the cells were re-suspended in 400 μL of staining buffer. The samples were acquired by BD FACSDiva™ on CANTOII™, then analyzed using FlowJo™ v10.

### Generation of the Steroid Acid-Peptide Conjugates

Steroid acid-peptide conjugates (e.g., CA-SV40 NLS) were synthesized as previously described in Beaudoin et al., 2016, in U.S. Pat. No. 11,291,717, and in PCT application publication number WO/2022/232945, unless otherwise indicated. For example, for CA-SV40 NLS, cholic acid was conjugated to the free amino group of the N-terminal cysteine residue of a 13-mer peptide (CGYGPKKKRKVGG; SEQ ID NO: 1) that comprises a nuclear localization signal from SV40 large T-antigen (SEQ ID NO: 2) flanked by linker amino acids. For cargo conjugations, cargoes were solubilized at 1-10 mg/mL in sterile PBS with or without other formulation components, but free of amine (NH₃) or sulfhydryl (SH) groups. The SM(PEG)₄ cross-linker was added to the reaction for 1 h using different molar excess ratios (10× for Cas9-NLS or Cas9-GFP cargoes; 50× for OVA cargoes). The free SM(PEG)₄ cross-linker was discarded by Centricon™ filtration and Sephadex™ column. Steroid acid-peptide conjugates were added in the same molar excess ratios and incubated for 1 h to obtain different amounts of steroid acid-peptide moieties per cargo. Free unconjugated steroid acid-peptide conjugates were removed by Centricon™ filtration and Sephadex™ column. Steroid acid-peptide-cargo conjugates were concentrated in sterile PBS to obtain final concentration 5-10 mg/mL as determined by UV absorbance.

To evaluate steroid acid-peptide-cargo loading, 10 μg of unconjugated or conjugated cargoes were separated under reducing conditions on a 12% polyacrylamide gel and stained with Coomassie brilliant blue R-250™ (Bio-Rad, Mississauga, ON, Canada). The migration distance in the gel relative to the blue dye front (Rf) was measured and the numbers of steroid acid-peptide moieties conjugated per cargo were estimated by reference to a logarithm plot of molecular weight versus 1/Rf for Kaleidoscope pre-stained standards (Bio-Rad) electrophoresed under identical conditions. In addition, Western blot analysis against the cargoes were performed to confirm the Coomassie results.

### DC2.4 Transfection and Assessment of Damaged Endosomes by Microscopy

For this assay, 15×10³ DC2.4 cells were seeded on a sterile cover slide in a 24-well plate. Two days following transfection of DC2.4 cells with the eGFP-hGal3 mammalian expression vector, 0.1 mg/mL of cargo was added to cells then incubated for 3 h at 37° C. The cells were then washed twice to remove excess protein prior to being mounted on a slide. The slides were viewed by fluorescent microscopy (Nikon, Eclipse™ Ti2-U) and the results analyzed using the ImageJ™ software.

### Monitoring Intracellular Cargo Degradation/Processing

To evaluate OVA degradation/processing, cells were incubated with 10 g/mL DQ™ OVA (with or without steroid acid-peptide modification) at 37° C. 30 minutes later, cells were washed, and regular media was added. At the end of the indicated incubation time, cells were collected and washed with cold PBS containing 2% FBS. Fluorescence was monitored by analyzing the cells by flow cytometry.

### Assessment of Intrinsic Tryptophan Fluorescence (ITF)

An Applied Photophysics (Leatherhead, Surrey, UK) Chirascan™ Q100 circular dichroism (CD) spectrometer was used for intrinsic tryptophan fluorescence (ITF) analysis and a VWR digital heatblock (Radnor, PA) was used for dry block temperature incubations. The Chirascan™ Q100 autosampler rack cooling system was used for all 4° C. incubations. Data was analyzed using MATLAB™ software (Natick, MA). Briefly, samples were removed from storage at −20° C. and allowed to equilibrate to room temperature. Samples were then diluted to 0.8 mg/mL in PBS from stock concentrations in the range of 4 to 5 mg/mL. Diluted samples were then analyzed for ITF without exposure to thermal stress (Native) or after ten minutes of thermal stress by dry block incubation. An aliquot of each diluted sample was incubated at 4° C., a second aliquot was incubated at 37° C., while a third aliquot was incubated at 80° C. BSA, diluted to 0.8 mg/mL, was included with the samples under each of the thermal conditions described above. All samples were re-equilibrated to room temperature after incubation. ITF Analysis was performed in 8 triplicates by excitation at 280 nm with an emission scan range of 200-600 nm with a bandwidth of 1.0 nm, a Time-per point of 1 s, and a Step of 0.5. The triplicate spectra were blank subtracted, averaged, and converted from units of mdeg to relative fluorescence intensity using MATLAB software. Diluted BSA solutions were assayed as controls preceding and following the sample sequence.

### Antigen Cross-Presentation Assay

To evaluate antigen cross-presentation, cells were seeded at 25×10³ cells per well in 24-well plates (Coming; Massachusetts, United States), then pulsed with antigens or antigen-containing mixtures at different concentrations for 3 h. At the end of the pulsing period, the cells were washed to remove excess antigen and co-cultured with 10⁶/mL CD8 T-cells purified from the spleens of OT-I mice using T-cell isolation kits according to the manufacturer's protocol. After 72 hours, supernatants were collected and used to quantify cytokine production by commercial enzyme-linked immunosorbent assays (ELISAs).

### Antigen-Presentation Assay Using the B3Z Reporter System

Various bile acid-NLS conjugates were screened using the B3Z reporter system. The B3Z cell line is a T-cell hybridoma specific for the H2-K^{b}-SIINFEKL complex. Once activated via its TCR, the LacZ reporter gene (under the NFAT promoter control) is expressed. Briefly, 1.5×10⁵ BMDCs or 2.5×10⁵ MSCs were co-cultured with 5×10⁴ B3Z cells treated with the mixing conditions of ovalbumin (OVA) and bile acid-NLS conjugates for overnight at 37° C. with 5% CO₂. The following day, all cells were washed twice with PBS (pH 7.4), and the cell pellets were lysed by adding 100 μL of a lysis buffer containing 0.15 mM chlorophenol red-beta-D-galactopyranoside (CPRG) substrate (Calbiochem, La Jolla, CA), 0.125% NP40 (EMD Sciences, La Jolla, CA), 9 mM MgCl₂ (Aldrich, USA) and 100 mM 2-mercaptoethanol in PBS. After a 5- or 24-h incubation at 37° C., absorbance was taken at 570 nm with 636 nm as the reference wavelength. For these experiments, OVA was re-suspended in PBS (pH 7.3) at 5-10 mg/mL. The different bile acid-NLS conjugates were re-suspended in H₂O at 10 mg/mL. Bile acid-NLS conjugate: antigen mixtures were prepared at different molar ratios according to Table 1.

TABLE 1
Molar Ratios of Bile Acid-NLS conjugate: OVA

| Molar ratio (bile acid- | 2.2:1 | | | | 22:1 |
|---|---|---|---|---|---|
| NLS conjugate:OVA) | (50 μmol/L) | 4:1 | 8:1 | 12:1 | (500 μmol/L) |
| OVA (mmol) | 2.27273 × 10⁻⁵ | 2.27273 × 10⁻⁵ | 2.27273 × 10⁻⁵ | 2.27273 × 10⁻⁵ | 2.27273 × 10⁻⁵ |
| | (1 mg) | (1 mg) | (1 mg) | (1 mg) | (1 mg) |
| Bile acid-NLS | 0.00005 | 9.09091 × 10−5 | 0.00018182 | 0.000272727 | 0.0005 |
| conjugate (mmol) | | | | | |

### Animals and Ethics

All female Balb/c and C57BL/6 (6-8 weeks old) mice were purchased from the Jackson Laboratory (Bar Harbor, ME, USA) and housed in a pathogen-free environment at the animal facility located at the Institute for Research in Immunology and Cancer (IRIC). All experimental procedures and protocols were approved by the Animal Ethics Committee (CDEA) of Universite de Montreal.

### Antibodies and Reagents

The flow-cytometry antibodies (CD44, CD45, CD73, CD90, H2-K^{b}, and I-A^{b}) were purchased from BD Biosciences (San Jose, CA, USA). OVA-AF647 and OVA-DQ® were purchased from Life Technologies (Waltham, MA USA) and used according to manufacturer's instructions. The annexin-V staining kit was purchased from Cedarlane (Burlington, ON, CANADA). Recombinant Cytochorme C was purchased from Sigma Aldrich (Oakville, ON, CANADA).

### Cell Lines

The EG.7 cell line used in this study was obtained from ATCC. The B3Z cells were maintained in Roswell Park Memorial Institute (RPMI) 1640 Medium supplemented with 10% fetal bovine serum (FBS). EG.7 cells were cultured RPMI 16400 supplemented with 2 g/L Glucose, 10% FBS, 50 U/mL Penicillin-Streptomycin, 2 mM L-glutamine, 10 mM HEPES, 1 mM Sodium Pyruvate, and 0.5 mM 0-Mercaptoethanol, and kept under selection using 80 mg/mL of G418. All cells were maintained at 37° C. in a 5% CO₂ incubator. All cell culture media and reagents were purchased from Wisent Bioproducts (St-Bruno, QC, Canada).

### Generation of BM-Derived MSCs

In order to generate Bone marrow (BM)-derived mouse MSCs, the femurs of 6-8-week-old female Balb/c or C57BL/6 mice were isolated and flushed with Alpha Modification of Eagle's Medium (AMEM) supplemented with 10% FBS and 50 U/mL Penicillin-Streptomycin in a 10 cm cell culture dish, then incubated at 37° C. Two days later, non-adherent cells were removed and the media replaced every 3 to 4 days until plastic-adherent cells reached 80% confluency. The generated cells were detached using 0.05% trypsin and expanded until a uniform MSC population was obtained. The generated MSCs were validated for their innate phenotype by flow-cytometry for the expression of CD44, CD45, CD73, and CD90. The cells were frozen in liquid nitrogen until use.

### Antigen Cross-Presentation Screening Assay Performed on Wild-Type MSCs

To assess cross-presentation assay, 25×10³ cells MSCs were seeded per well in 24-well plate then pulsed with steroid acid peptide-conjugated for 6 h admixed with OVA. At the end of the pulsing period, the cells were washed to remove excess antigen then 5×10⁴ B3Z cells. The cells were incubated for 17-19 hours prior to their lysis and incubation for another 4-6 hours at 37° C. with a CPRG solution. The optical density signal was detected at wavelength 570 using a SynergyH1™ microplate reader (Biotek, Winooski, VT, United States).

### Monitoring Antigen Uptake and Processing

To evaluate OVA uptake, MSCs were first treated with 1 g/mL of OVA-AF647 admixed with CA-hnRNPA1 for 1 hour at 37° C. then assessed for their fluorescence intensity by flow-cytometry. To evaluate antigen processing, MSCs were incubated with 10 g/mL OVA-DQ® admixed with A1 at 37° C. Half an hour later, cells were washed, and regular media added. At the end of the indicated incubation time, cells were collected and washed with cold PBS containing 2% FBS. Fluorescence was monitored by flow cytometry.

### Assessing Endosomal Escape

Endosomal leakage was assessed using an apoptosis assay. Briefly, 10′ MSCs were first supplemented with 10 mg/mL of exogenous rCyt-C for 6 h at 37° C. in the presence or absence of CA-hnRNPA1 (50 μM). Once the incubation period completed, the cells were collected using Accutase®, washed with ice cold PBS, then stained for Annexin-V according to manufacturer's instructions prior to analysis using BD FACS Diva™ on CANTOII™.

### Turbidity Assay

For the turbidity assay to track protein aggregation, OVA (1 mg/mL) and CA-hnRNPA1 (50 μM) were diluted in serum-free AMEM. 100 μL of each sample were added to a polystyrene flat bottom 96-well plate (Coming). The wavelength for measurement was defined according to examination of the absorbance spectra of the buffer (serum-free AMEM) in which no significant peak was observed. Thus, turbidity was assessed at 420 nm using a Synergy H1 microplate reader (BioTek). Plates were incubated at 37° C. and shaken for 5 s before each reading, that was taken in every 15 minutes. The experiment was conducted 4 times and in each 6 technical replicates were performed.

### Cytokine and Chemokine Analysis

For cytokine and chemokine profiling, 15 cm cell culture dishes containing 80-90% confluent MSCs were grown in serum-free AMEM for 24 h at 37° C. and 5% CO₂. MSCs were then treated with 50 μM of CA-hnRNPA1 in serum-free AMEM for 6 h. The supernatant was collected and fresh serum-free AMEM was replenished, without CA-hnRNPA1. After 24 h of the initial CA-hnRNPA1 treatment, the supernatant was collected and gathered with the previous one collected. Collected supernatants were then concentrated using the Amicon Ultra-4™ centrifugal filters (3000 NMWL) for 1 h at 4° C. Collected concentrates (80×) were then frozen at −80° C. until shipped to EveTechnologies™ (Calgary, AB, Canada) for cytokine/chemokine assessment by Commerc™.

### Therapeutic Vaccination

For therapeutic vaccination, female C57BL/6 mice (n=10/group) received a SC injection of 5×10⁵ EG.7 cells at day 0. Five days later (appearance of palpable tumors ˜35-50 mm³), mice were SC-injected with 5×10⁵ CA-hnRNPA1+OVA-pulsed MSCs (two injections 1 week apart). Control animals received 5×10⁵ tumor cells alone. Treated animals were followed thereafter for tumor growth. For therapeutic vaccination in combination with the immune-checkpoint inhibitors (anti-PD-1), mice received SC-injections of the antibody or its isotype at 200 g/per dose every 2 days for a total of 6 doses over two weeks. A similar approach was conducted for allogeneic dosing vaccination in C57BL/6 mice but using Balb/c-derived MSCs.

### RNA-Seq and Bioinformatic Analysis

For RNA-seq, control MSCs or MSCs treated with CA-hnRNPA1 alone or CA-hnRNPA1+OVA for 6 h were used to extract RNA a commercial RNA extraction kit. Quantification of total RNA was made by QuBit™ (ABI) and 500 ng of total RNA was used for library preparation. Quality of total RNA was assessed with the BioAnalyzer™ Nano (Agilent) and all samples had a RIN above 8. Library preparation was done with the KAPA™ mRNAseq stranded kit (KAPA, Cat no. KK8420). Ligation was made with 9 nM final concentration of Illumina index and 10 PCR cycles was required to amplify cDNA libraries. Libraries were quantified by QuBit and BioAnalyzer. All libraries were diluted to 10 nM and normalized by qPCR using the KAPA library quantification kit (KAPA; Cat no. KK4973). Libraries were pooled to equimolar concentration. Sequencing was performed with the Illumina Hiseq2000 using the Hiseq™ Reagent Kit v3 (200 cycles, paired-end) using 1.7 nM of the pooled library. All Fastq files (strand-specific sequencing, N=4 per group) were aligned to GRCm38 (mouse genome Ensembl release 102) with STAR (v2.7). Raw reads mapping to genomic features (summarized per gene) were extracted with featureCounts (strand specific option). Expression matrices were filtered, genes with very low counts were removed and protein coding genes were kept for further analyses. Gene expression in both CA-hnRNPA1—and CA-hnRNPA1+OVA-treated MSCs were compared to BM-Derived MSC controls with DESeq2™ to generate a ranked list of differentially expressed genes based on the log 2 fold change. Gene set enrichment on either ranked lists of genes, or a number of significantly up- or down-unregulated genes perturbed by CA-hnRNPA1 alone or admixed with CA-hnRNPA1 variant compared to MSC controls were performed using the Reactome collection of pathways. The variance stabilizing transformation was applied to gene expression matrices prior to visualization. If not mentioned in the text, significance threshold is set to 5% after p-value adjustment with the Benjamini-Hochberg method to control for false positives among differentially expressed genes (DEGs). All custom scripts including prediction of putative targets were written in R programming and statistical language. Data visualization was made with ggplot2, enrichplot, Upset plots and Pheatmap R functions.

### Statistical Analyses

p-values were calculated using one-way analysis of variance (ANOVA). Results are represented as average mean with standard deviation (S.D.) error bars and statistical significance is represented with asterisks: * p<0.05, ** p<0.01, *** p<0.001.

### Example 2: Conjugation of Cas9-NLS Cargo with CDCA-SV40 Results in Robust Nuclear Delivery in HEK293 Cells

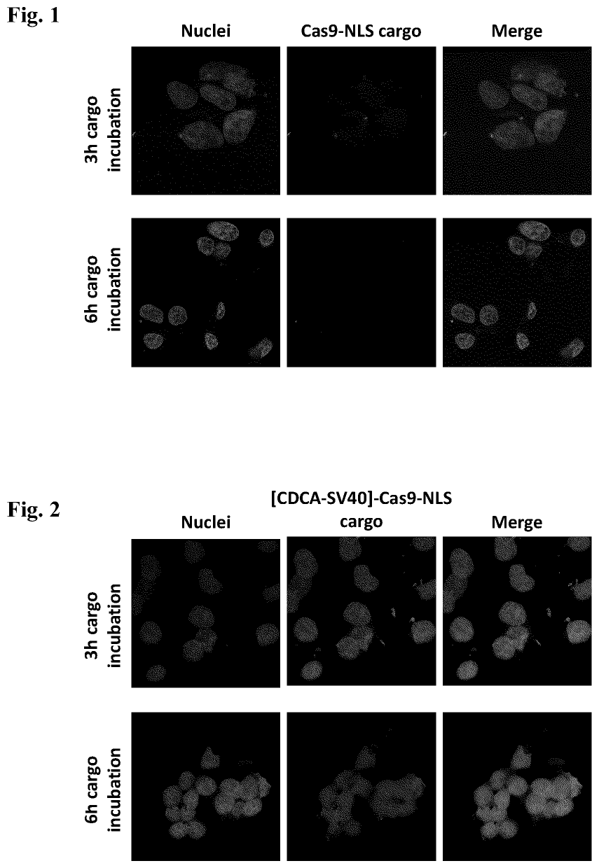

Recombinant Cas9-NLS protein conjugated with a molar excess of CDCA-SV40 NLS steroid acid-peptide moieties ([CDCA-SV40]-Cas9-NLS) was produced as described in Example 1. HEK293 cells were incubated with 2 μM of either unconjugated Cas9-NLS cargo or [CDCA-SV40]-Cas9-NLS cargo for 0, 3 or 6 hours at 37° C., and then intracellular Cas9-NLS delivery was assessed by fluorescence microscopy as described in Example 1. Representative microscopy images for cells incubated with unconjugated Cas9-NLS or conjugated [CDCA-SV40]-Cas9-NLS as cargo are shown in FIG. 1 and FIG. 2, respectively. The results in FIG. 1 show that incubation of HEK293 cells with unconjugated Cas9-NLS as cargo resulted in only low levels of intracellular cargo delivery observable by fluorescence microscopy, even after 3 and 6 h. The mostly punctate pattern observed for the Cas9-NLS cargo suggested that the intracellular delivery was mainly endosomal. In contrast, the results in FIG. 2 show that incubation of HEK293 cells with [CDCA-SV40]-Cas9-NLS resulted in a strikingly high level of intracellular delivery at 3 and 6 h. The pattern was mostly nuclear, suggesting that conjugated cargo avoided endosomal entrapment and was able to reach the nucleus.

### Example 3: Intracellular Delivery of [CA-SV40]-OVA is Associated with Endosomal Membrane Disruption in DC2.4 Cells

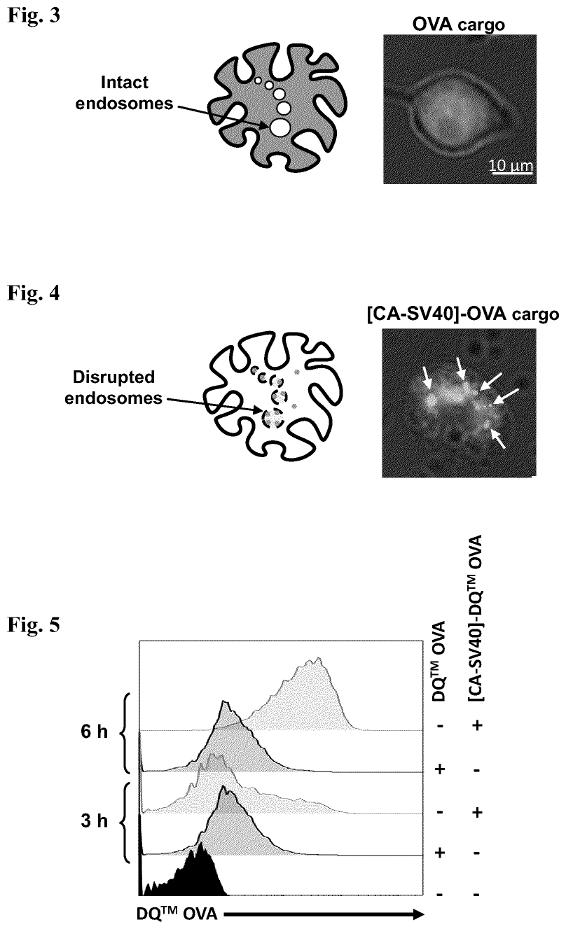

A GFP-galectin-3 (GFP-Gal3) based detection system was utilized to explore the effect on endosomal membranes following intracellular delivery of steroid acid-peptide-conjugated cargoes. Briefly, Gal3 is a cytosolic protein that exhibits high affinity towards β-galactoside sugars, which are normally present on the cell surface, Golgi apparatus, and in the lumen of endocytic compartments (i.e., compartments sequestered from the cytosol). When expressed under normal conditions, Gal3 is evenly distributed across the cytosol but disruption of endosomal membranes allows Gal3 to access and bind luminal glycoproteins. We thus transiently transfected the murine dendritic cell line DC2.4 with a construct expressing Gal3 fused to enhanced green fluorescent protein (eGFP-Gal3). Two days later, DC2.4 cells were incubated with 0.1 mg/mL of either unconjugated OVA cargo or [CA-SV40]-OVA conjugated cargo for 3 h at 37° C. and the cells were observed by fluorescent microscopy. As shown in FIG. 3, the eGFP-Gal3 marker remained cytosolic following incubation with unconjugated OVA cargo. In contrast, the eGFP-Gal3 marker became punctate following intracellular delivery of the [CA-SV40]-OVA conjugated cargo, suggesting that the avoidance of endosomal entrapment by conjugated cargoes is associated with disruption of endosomal membranes (FIG. 4).

### Example 4: [CA-SV40]-OVA Cargoes are Metabolizable by Intracellular Proteases in Primary Dendritic Cells

The fluorogenic substrate DQ™ ovalbumin (DQ™ OVA) was employed to study the intracellular fate of steroid acid-peptide-conjugated cargoes. Briefly, while a strong fluorescence quenching effect is observed when the DQ™ OVA substrate remains intact, hydrolysis of DQ™ OVA into single dye-labeled peptides by proteases relieves this quenching, thereby producing brightly fluorescent products. Recombinant DQ™ OVA conjugated with a molar excess of CA-SV40 NLS steroid acid-peptide moieties ([CA-SV40]-DQ™ OVA) was produced as described in Example 1. Primary bone marrow-derived DCs were incubated with either DQ™ OVA unconjugated cargo or [CA-SV40]-DQ™ OVA conjugated cargo for 3 or 6 h at 37° C. Cells were then collected and fluorescence was monitored by flow cytometry. As shown in FIG. 5, a sharp increase (i.e., right shift) in fluorescence was observed at six hours for cells incubated with the [CA-SV40]-DQ™ OVA cargo, which was not observed for cells incubated with the unconjugated DQ™ OVA cargo. These results suggest that conjugation of proteinaceous cargoes with steroid acid-peptide moieties are metabolizable by intracellular proteases.

### Example 5: Coincubation of Unconjugated OVA Cargo with CA-HnRPA1 Steroid Acid-Peptide is Associated with Increased Intracellular Delivery and Endosomal Escape in Mesenchymal Stromal Cells

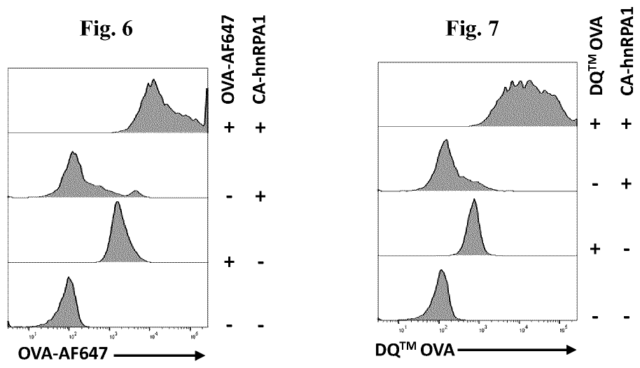

Mesenchymal stromal cells (MSCs) were incubated with either unconjugated fluorescently labeled OVA-AF647 alone or mixed with 45 μM of CA-HnRPA1 NLS steroid acid-peptide for nine hours and then intracellular OVA-AF647 delivery was assessed by flow cytometry as described in Example 1. As shown in FIG. 6, coincubation of the unconjugated OVA-AF647 with CA-HnRPA1 resulted in increased intracellular cargo delivery as compared to incubation with the cargo alone. The experiment was repeated with DQ™ OVA as cargo to evaluate the intracellular fate of the cargo delivered upon coincubation with CA-HnRPA1 NLS, as described in Example 4. As shown in FIG. 7, a sharp increase (i.e., right shift) in fluorescence was observed at nine hours for cells coincubated with both unconjugated cargo and CA-HnRPA1 NLS, consistent with endosomal escape and eventual processing of the cargo by intracellular proteases.

### Example 6: Coincubation of Cytochrome C Cargo with CA-SV40 NLS Steroid Acid-Peptide is Associated Enhanced Cytosolic Delivery and Induction of Apoptosis in EL4 Cells

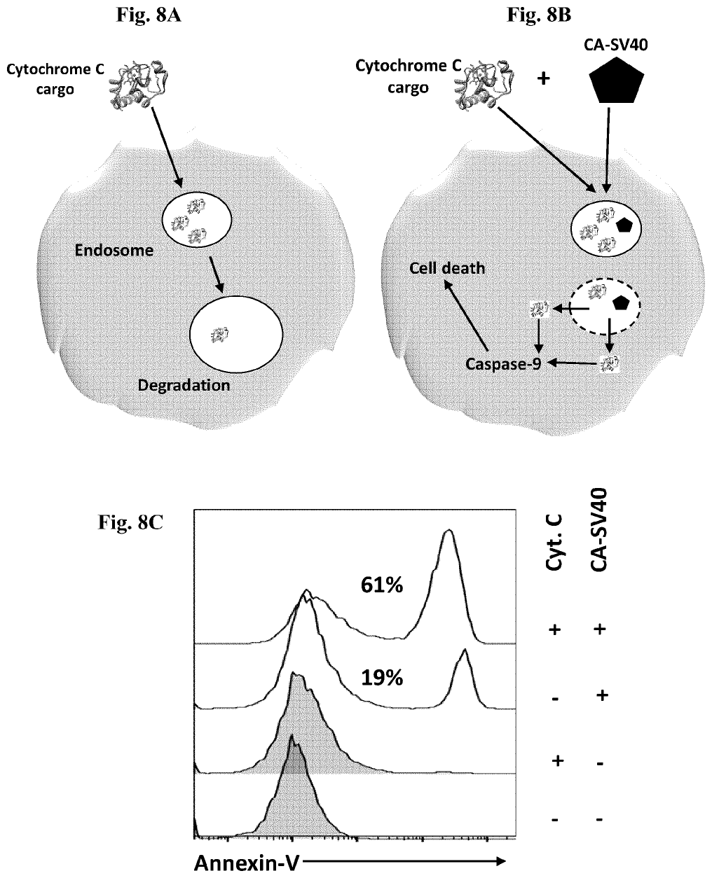

Cytochrome C is a protein that is normally entrapped in the mitochondria but its release into the cytosol in known to induce cell death. An experiment was performed in which EL4 cells were incubated with recombinant cytochrome C either alone (FIG. 8A) or mixed with CA-SV40 NLS (47 μM) (FIG. 8B). Successful delivery of cytochrome C to the cytosol in its active form was assessed by measuring cell death (annexin V binding) via flow cytometry. Interestingly, incubation with cytochrome C alone did not trigger cell death, whereas coincubation with CA-SV40 increased cell death by more than three-fold (19% to 61%, FIG. 8C). These results suggest that the steroid acid-peptide conjugate, CA-SV40 NLS, facilitates cargo delivery to the cytosol in its biologically active form. The observation that the CA-SV40 NLS conjugate alone resulted in 19% cell death suggests that other steroid acid-peptide conjugates having reduced cytotoxicity may be advantageously considered.

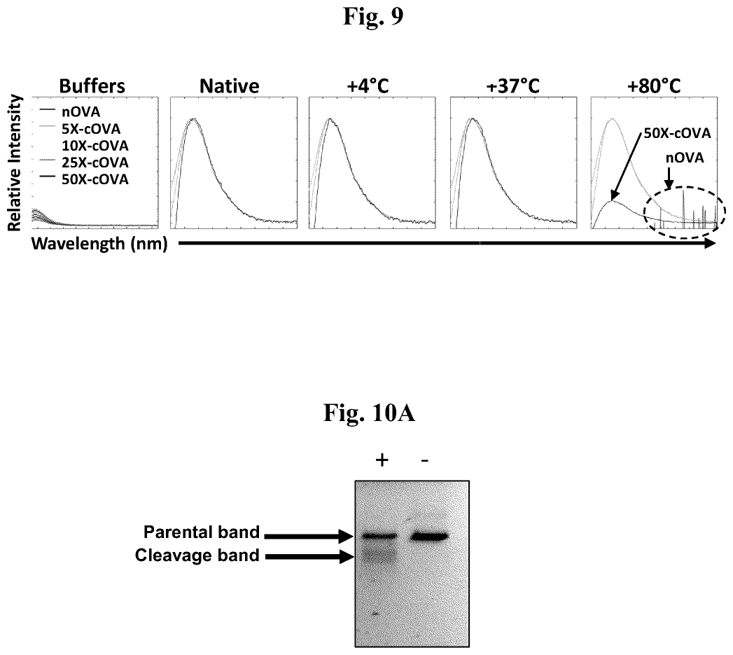

### Example 7: Biochemical Characterization of [CA-SV40]-OVA

[CA-SV40]-OVA prepared as described in Example 1 using different molar excess ratios of OVA cargo to CA-SV40 NLS conjugate. SDS-PAGE followed by Coomassie staining revealed that [CA-SV40]-OVA prepared using a 25× molar excess of CA-SV40 NLS had an average of about four [CA-SV40] moieties conjugated per OVA, corresponding to a MW increase of about 8.6 kDa compared to unconjugated OVA. [CA-SV40]-OVA prepared using a 50× molar excess of CA-SV40 NLS had an average of about eight [CA-SV40] moieties conjugated per OVA, corresponding to a MW of about 19.2 kDa. Furthermore, to assess the overall stability of [CA-SV40]-OVA, ITF analysis was conducted to measure its unfolding following thermal stress. In this assay, changes in peak shifts or intensities are indicative of unfolding as polypeptide residues may become solvent-exposed and undergo change in orientation (FIG. 9). When different OVA: CA-SV40 NLS ratios were assayed under native or thermally variable conditions, naked or unconjugated OVA (FIG. 9, “nOVA”) underwent complete denaturation at 80° C. along with partial reduction in peak intensity observed for OVA conjugated with a 50× molar excess of CA-SV40 NLS (FIG. 9, “50×-cOVA”). No changes in ITF spectral measures were observed for the other conjugated OVA samples suggesting that conjugation with the [CA-SV40] moieties greatly increased cargo stability.

### Example 8: Bile Acid-NLS Moieties Increase Intracellular Delivery of Cas9-GFP in JIMT-1 Cells in the Presence or Absence of a Lipid-Based Transfection Agent

A Cas9-GFP fusion cargo protein was conjugated to (e.g., [CA-SV40]-Cas9-GFP), or mixed with (e.g., Cas9-GFP+[Bile acid-NLS]), different bile acid-NLS moieties and then evaluated for intracellular delivery as described in Example 1. Delivery experiments were performed in JIMT-1 cells, which are generally considered difficult to transfect, and intracellular cargo delivery was measured via flow cytometry based on GFP fluorescence. Briefly, JIMT-1 cells were co-incubated with 5 μg (0.0257 nmol) Cas9-GFP cargo and 0.275 μmol different bile acid-NLS moieties for 48 hours and then intracellular Cas9-GFP delivery was assessed with a Biotek Spectrometer (final volume 2 mL). The results are shown in Table 2 with GFP fluorescence values being normalized to that of cells incubated with the unconjugated cargo alone (Cas9-GFP alone). The results in Table 2 suggest that bile acid-NLS moieties can increase intracellular delivery of proteinaceous cargoes, even in some cells/cell lines traditionally considered difficult to transfect. The increase in intracellular delivery was observed with the moieties being conjugated to or simply mixed with the cargoes. Fluorescence microscopy experiments in live cells confirmed that the Cas9-GFP cargoes (which were engineered to contain an NLS) were successfully delivered to the nucleus of JIMT-1 cells (data not shown).

TABLE 2
Cas9-GFP delivery by bile acid-NLS moieties

| Cargo/bile acid-NLS moiety | Relative intracellular GFP fluorescence |
|---|---|
| Cas9-GFP alone | 1.0 |
| [CA-SV40]-Cas9-GFP | 1.3 |
| Cas9-GFP + [CA-GWG-SV40] | 1.5 |
| Cas9-GFP + [CA-cMyc] | 4.1 |

To assess whether the enhanced intracellular delivery conferred by bile acid-NLS moieties is compatible with other delivery technologies, a delivery experiment in JIMT-1 cells was performed using Lipofectamine™ CRISPRMAX™ Cas9 Transfection Reagent (Thermo Fisher Scientific) according to the manufacturer's protocol, except that the cargo was Cas9-GFP alone or conjugated to/mixed with different bile acid-NLS moieties. The results are shown in Table 3 with GFP fluorescence values being normalized to that of cells incubated with the unconjugated cargo/transfection reagent alone (Cas9-GFP alone). The results in Table 3 suggest that bile acid-NLS moieties can increase intracellular delivery of proteinaceous cargoes in the context of lipid-based delivery systems. The increase in intracellular delivery was observed with the moieties being conjugated to or simply mixed with the cargoes before formulation with the transfection reagent. Fluorescence microscopy experiments in live cells confirmed that the Cas9-GFP cargoes were successfully delivered to the nucleus of JIMT-1 cells (data not shown). Strikingly, mixture with the moiety CA-HuR resulted in a 17-fold increase in intracellular Cas9-GFP delivery as compared to Cas9-GFP/transfection reagent alone.

TABLE 3
Cas9-GFP delivery by bile acid-NLS
moieties in the presence of Lipofectamine

| | Relative intracellular |
|---|---|
| Cargo/bile acid-NLS moiety | GFP fluorescence |
| Cas9-GFP alone | 1.0 |
| [CA-SV40]-Cas9-GFP | 2.2 |
| Cas9-GFP + [CA-GWG-SV40] | 5.0 |
| Cas9-GFP + [CDCA-NLS1 RPS17] | 1.5 |
| Cas9-GFP + [CDCA-NLS3 RPS17] | 1.4 |
| Cas9-GFP + [CA-cMyc] | 1.9 |
| Cas9-GFP + [CA-cMyc] | 2.4 |
| Cas9-GFP + CA-HuR | 17.0 |

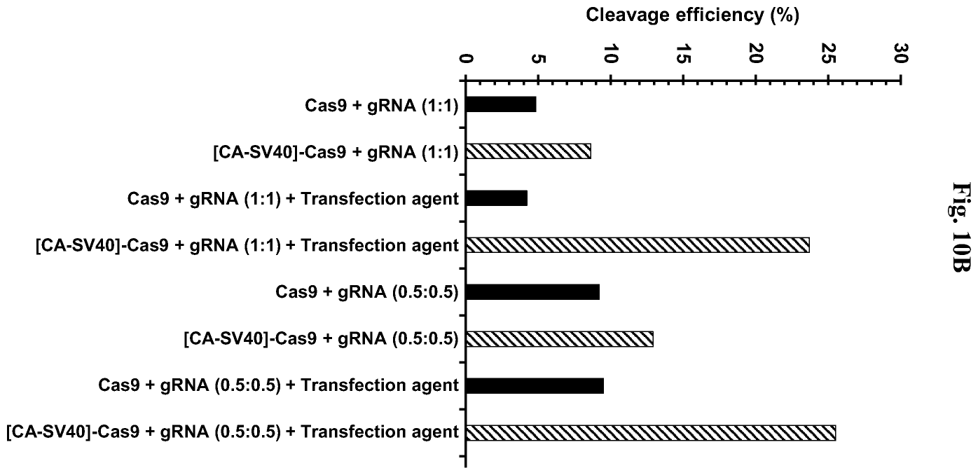

To assess whether Cas9 retains endonuclease activity following conjugation with bile acid-NLS moieties, a delivery experiment was performed by treating JIMT-1 cells with Cas9 complexed with TrueGuide™ CDK4 gRNA (Invitrogen). Endonuclease activity was assessed following delivery using the GeneArt™ Genomic Detection Kit (Cat. No: A24372, Life Technologies), with positive endonuclease activity being observable by the detection of genomic DNA cleavage products, as shown using the manufacturer's positive (+) and negative (−) controls in FIG. 10A. The results shown in FIG. 10B compare the cleavage activities of unconjugated Cas9 with [CA-SV40]-Cas9-GFP, with or without Lipofectamine™ CRISPRMAX™ Cas9 Transfection Reagent. The ratios in parentheses refer to the molar ratios between Cas9 and gRNA, wherein “1:1” means that the concentrations recommended by the manufacturer were used, while “0.5:0.5” means that half of the manufacturer's recommended concentrations were used. Cleavage efficiency (%) was calculated according to the manufacturer's instructions. The results in FIG. 10B show that [CA-SV40]-Cas9 RNP (striped bars) not only retained cleavage activity, but resulted in increased cleavage over their corresponding Cas9 RNP controls (solid bars). Interestingly, transfection with [CA-SV40]-Cas9 RNP in the absence of transfection reagent yielded cleavage efficiencies higher than those obtained from transfection with unconjugated Cas9 RNP in the presence of transfection reagent.

### Example 9: Bile Acid-NLS Moieties Increase Nuclear Delivery of Plasmid DNA

Further delivery experiments were performed to assess the impact of bile acid-NLS moieties on cytosolic/nuclear delivery of polynucleotide cargoes. Poly-D-lysine (“poly-K”; MW: 110 kDa, Thermo Fisher Scientific) was conjugated with a 10-fold molar excess of [CA-SV40] moieties as described in Example 1. Plasmid DNA encoding GFP was used as cargo, with cytosolic/nuclear delivery being evaluated by flow cytometry based on GFP fluorescence. Briefly, plasmid DNA complexes were prepared by adding, dropwise and with constant mixing, 7.5 μg poly-K or 5 μg of [CA-SV40]-poly-K in 0.3 mL of serum-free DMEM to 8 μg plasmid DNA in 0.7 mL of serum-free DMEM (NH₃:phosphate=2:1). The mixed solutions were kept for 30 min at 20° C. before use. HEK293 cells were seeded (day 0) into wells of culture plates. On day 1, the medium was removed and 1 mL of a solution containing a plasmid/poly-K complexes in serum-free DMEM was added. After 6 h and 24 h incubation at 37° C. in a humidified atmosphere (95% air, 5% CO₂), 1 mL of complete medium was added and cells were further incubated at 37° C. in 0.5 mL of the relevant complete culture medium for another 24 h before the cells were collected and subjected to flow cytometry analysis. The results shown in Table 4 demonstrate the ability of bile acid-NLS moieties to delivery polynucleotide cargoes intracellularly to the cytosol/nucleus.

TABLE 4
Nuclear delivery of plasmid DNA by bile acid-NLS moieties

| | GFP fluorescence |
|---|---|
| Cargo | (geometric mean: GFP-A) |
| GFP plasmid alone | 17.7 |
| GFP plasmid/poly-K | 19.3 |
| GFP plasmid/[CA-SV40]-poly-K | 53.6 |

### Example 10: Enhanced Intracellular Delivery and Antigen Presentation in BMDCs by SV40NLS Conjugated to Different Bile Acids

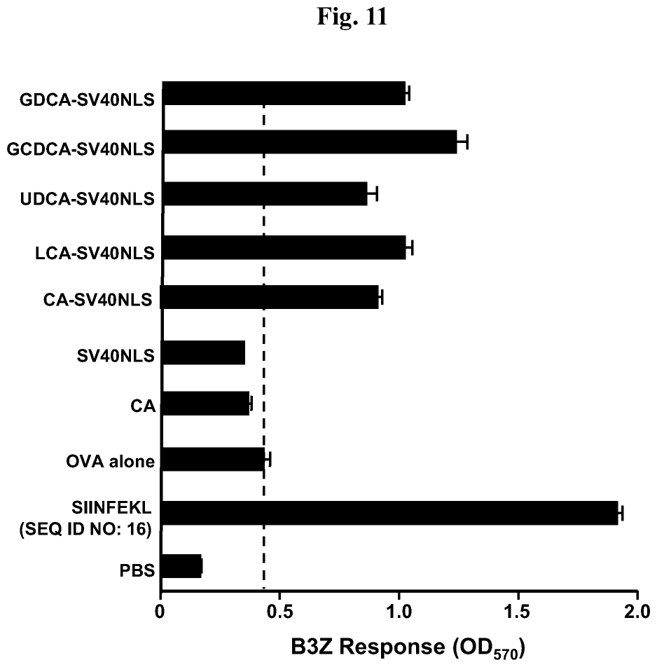

Variants of CA-SV40NLS were synthesized in order to explore structure-activity relationships relating to the antigen cross-presentation enhancing activity observed for this conjugate, and therefore a measure of antigen/cargo delivery. More particularly, conjugates having different bile acids conjugated to the SV40NLS peptide (SEQ ID NO: 1) were synthesized and their effect on antigen presentation was evaluated by using the B3Z reporter system with the OVA antigen as described in Example 1. The results in FIG. 11 show that increased antigen cross-presentation was observed in BMDCs when OVA was mixed with the CA-SV40NLS conjugate as compared to the OVA antigen alone (“OVA alone”; dashed line). These results were consistent with those observed using an OT-I CD8 T cell-based assay (data not shown). Interestingly, comparable or higher antigen cross-presentation to CA-SV40NLS was observed when cholic acid was replaced with the bile acids: glycodeoxycholic acid (GDCA), glycochenodeoxycholic acid (GCDCA), ursodeoxycholic acid (UDCA), and lithocholic acid (LCA). In FIG. 11, no increase in antigen cross-presentation in BMDCs over the antigen alone (“OVA”) was observed when OVA was mixed with either unconjugated cholic acid (“CA”) or SV40NLS peptide (“SV40NLS”), although lower sensitivity of the B3Z reporter system as compared to the OT-I CD8 T cell-based assay may have been a factor. Interestingly, subsequent assays using the same B3Z reporter system revealed up to about a 30% increase in B3Z response (OD₅₇₀) when OVA was mixed with unconjugated glycoursodeoxycholic acid (GUDCA; 22:1) over the OVA alone (data not shown). Furthermore, the immunogen enhancer activity of GUDCA was observed at all GUDCA: OVA molar ratios tested (i.e., 2:1, 4:1, 8:1, 12:1 and 22:1).

### Example 11: Enhanced Intracellular Delivery and Antigen Presentation in Anti-Presenting Cells Upon Admixture with Different NLS Peptides Conjugated to Cholic Acid

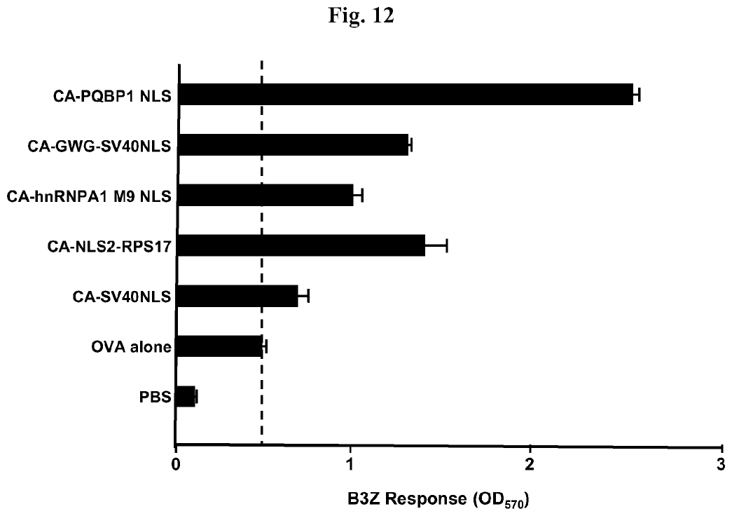

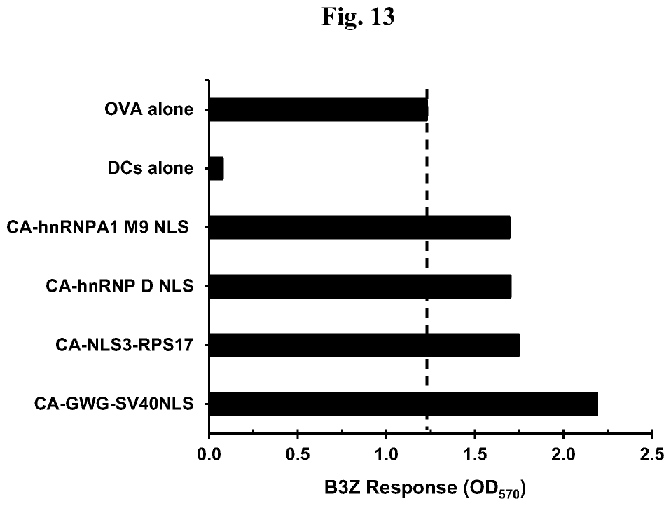

Further variants of CA-SV40NLS were synthesized in which the SV40NLS peptide was replaced with peptides comprising other NLS's (Table 5) and the antigen presentation activities of the CA-NLS peptide conjugates were evaluated using the B3Z reporter system as described in Example 1. The following conjugate: antigen molar ratios were tested for each conjugate: 2:1, 4:1, 8:1, 12:1 and 22:1. The results in FIGS. 12-15 compare the antigen presentation activities of different conjugates at the conjugate: antigen ratio that yielded the highest B3Z response (OD₅₇₀) for that conjugate.

TABLE 5
NLS peptides characterized in FIGs. 12-17

| | | | SEQ |
|---|---|---|---|
| | Peptide | | ID |
| NLS type | name | Peptide sequence | NO: |
| Classical | SV40NLS | CGYGPKKKRKVGG | 1 |
| NLS | | | |
| (K-K/R- | GWG-SV40NLS | CGWWGYGPKKKRKVGGWWG | 3 |
| X-K/R) | | | |
| | hnRNPA1 | CSNFGPMKGGNFGGRSSGPY | 4 |
| | M9 NLS | | |
| | hnRNP D NLS | CSGYGKVSRRGGHONSYKPY | 5 |
| | PQBP-1 NLS | CADREEGKERRHHRREELAPY | 7 |
| PY/G-NLS | hnRNP M NLS | CNEKRKEKNIKRGGNRFEPY | 6 |
| (hydro- | cMyc NLS | CGYGPAAKRVKLDGG | 12 |
| phobic | HUR NLS | CGRFSPMGVDHMSGLSGVNVPG | 13 |
| & basic) | Tus NLS | CGYGKLKIKRPVKGG | 14 |
| | NLS2-RG | CNKRVCEEIAIIPSKKLRNK | 8 |
| | Domain | GSGRIQRGPVRGIS | |
| | RRPS17 | | |
| Ribosomal | NLS1 RPS17 | CMGRVRTKTVKKAAGG | 15 |
| NLS | NLS2 RPS17 | CNKRVCEEIAIIPSKKLRNK | 10 |
| | NLS3 RPS17 | SKKLRNKIAGYVTHLMKRI | 11 |

The results in FIGS. 12-15 generally show that increased antigen presentation can be achieved by exposing antigen-presenting cells to the antigen in the presence of cholic acid conjugated to peptides comprising nuclear localisation signals of different types and having different amino acid sequences.

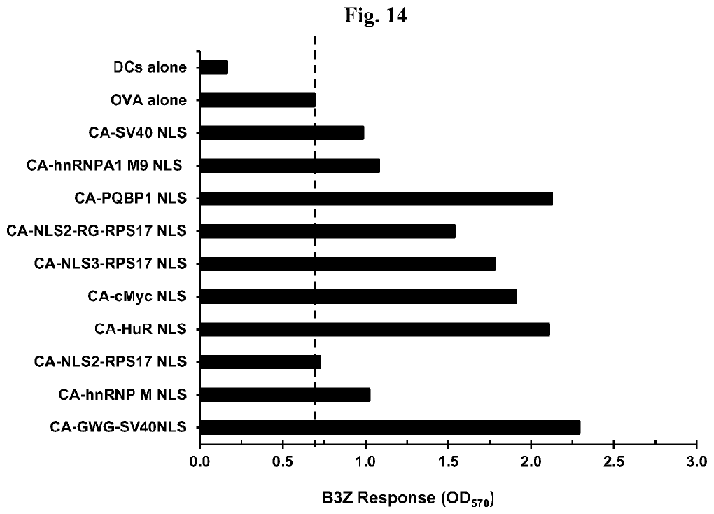

Using BMDCs as antigen presenting cells, the glutamate-rich peptide PQBP-1 NLS was associated with strikingly high antigen-presentation activity (FIGS. 12 and 14). Furthermore, NLS2-RG Domain RPS17, NLS3-RPS17, cMyc NLS, and HuR NLS peptides were also associated with high antigen presentation activity. Interestingly, the peptide GWG-SV40NLS was associated with higher antigen-presentation activity than SV40NLS, suggesting that the addition of flanking aromatic amino acids (WW or GWWG) was beneficial for activity (see FIGS. 12-14). Similar results were observed using a DC cell line (DC2.4) as antigen presenting cells.

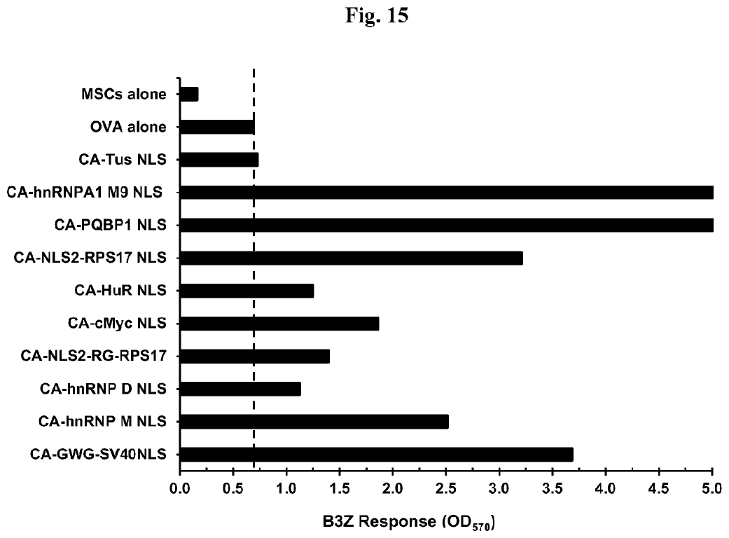

Using a cross-presenting cell line of MSCs (i.e., immortalized MSCs genetically engineered to possess cross-presenting capabilities, “cpMSCs”) as antigen presenting cells, various cholic acid peptide conjugates enhanced antigen presentation of OVA (FIG. 15). Similar to BMDCs, cholic acid-peptide conjugates comprising PQBP-1 NLS, HuRNLS, and GWG-SV40NLS were associated with strikingly high antigen-presentation activity, as compared to OVA alone or OVA mixed with CA-SV40NLS. CA-hnRNPA1 M9 NLS (“CA-hnRNPA1”) was also shown to highly enhance cross presentation in the MSC cell line.

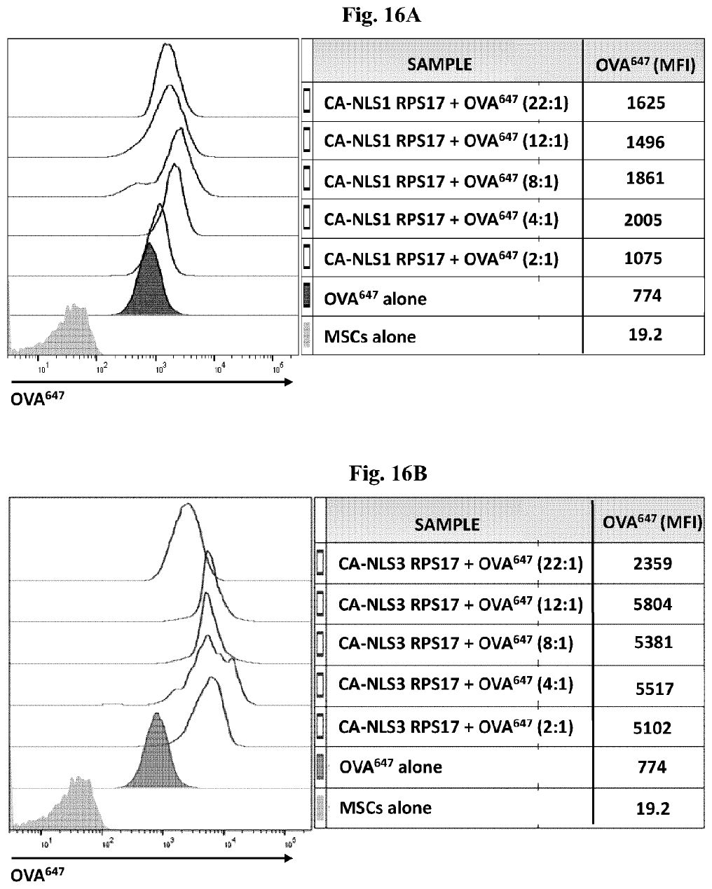

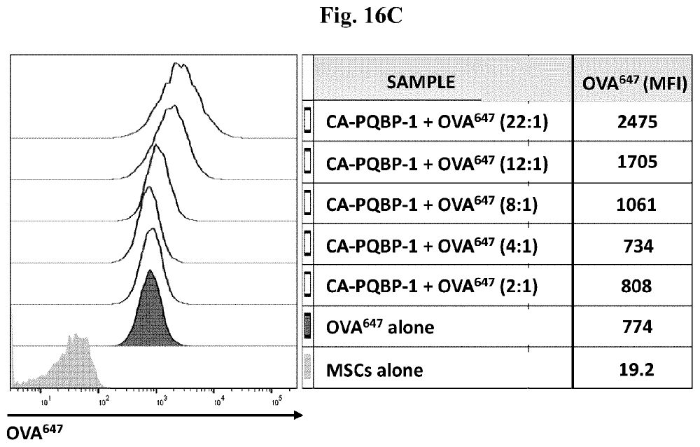

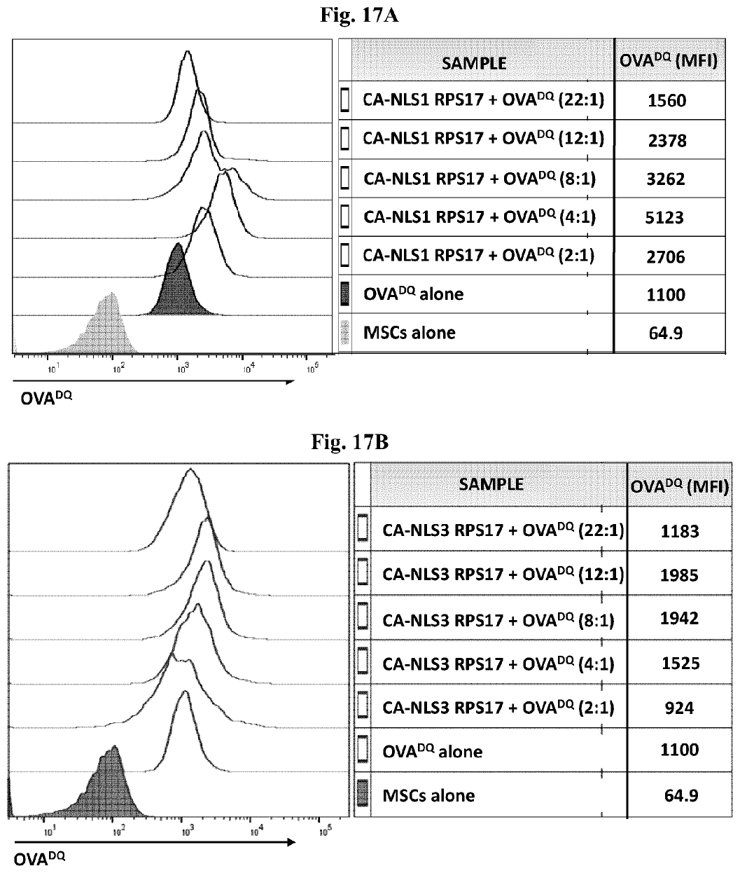

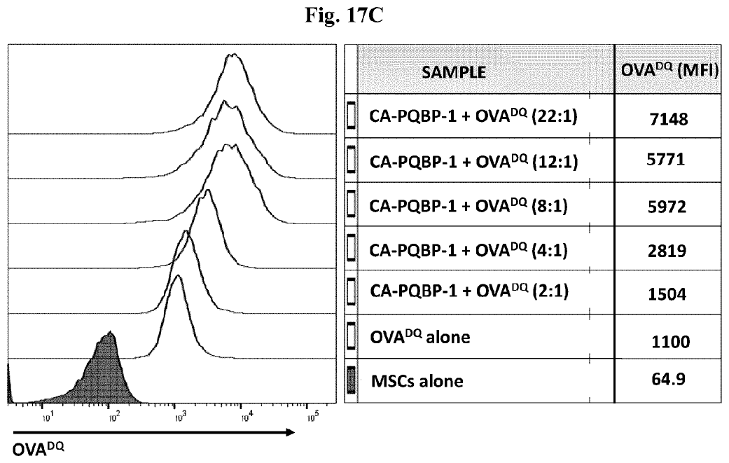

To further dissect the effect of bile acid peptide conjugates on antigen presentation, antigen internalization and processing were evaluated. cpMSCs were pulsed with OVA-labelled with AF647 in the presence of various molar ratios of different bile acid peptide conjugates, NLS1-RPS17 [FIG. 16A]; NLS3 RPS17 [FIG. 16B]; PQBP-1 [FIG. 16C]; and hnRNPA1 M9 NLS [FIG. 6]) and fluorescence was assessed by flow cytometry. Bile acid conjugates were shown to enhance OVA internalization, generally with increasing ratios. OVA processing was assessed by pulsing cpMSCs with DQ™-Ovalbumin (OVA-DQ) in the presence of the same bile acid peptide conjugates as in FIGS. 6 and 16. Bile acid conjugates NLS1-RPS17 [FIG. 17A]; NLS3 RPS17 [FIG. 17B]; PQBP-1 [FIG. 17C]; and hnRNPA1 M9 NLS [FIG. 7]) were shown to enhance OVA processing, generally with increasing ratios.

In summary, these data demonstrate the versatility and capability of bile acid peptide conjugates in enhancing cargo/antigen delivery, processing, and presentation.

### Example 12: Enhanced Intracellular Delivery and Antigen Cross Presentation in Wild-Type MSCs Upon Admixture with Steroid Acid-Peptide Conjugates

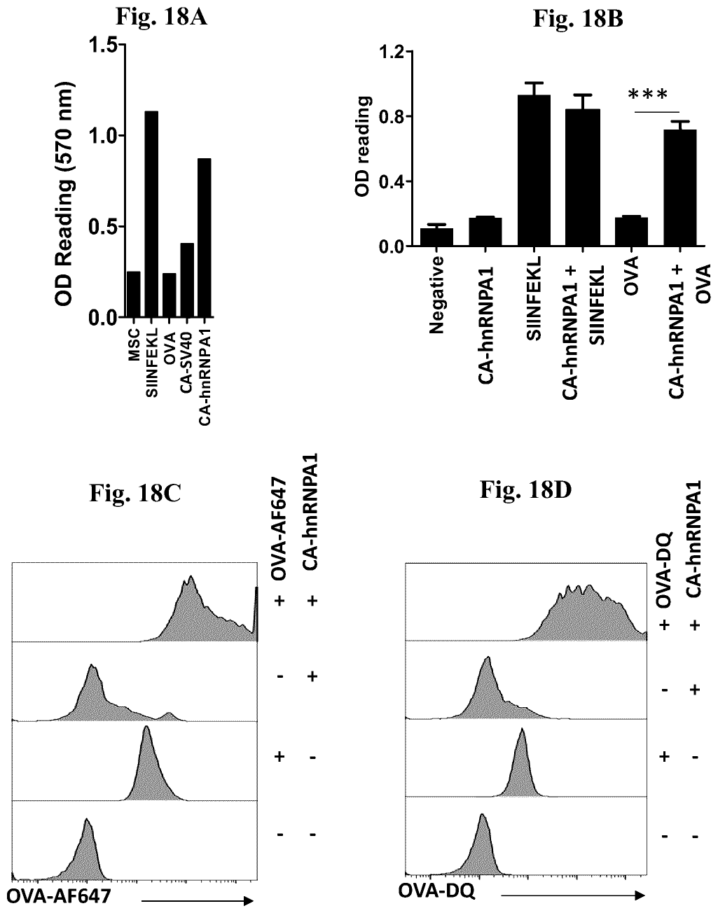

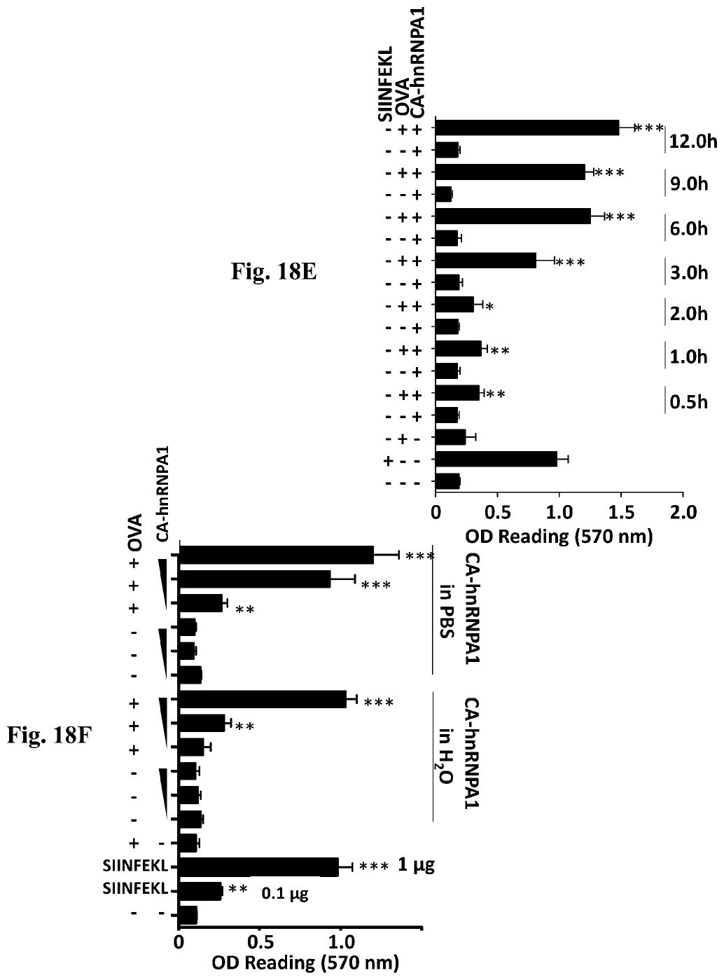

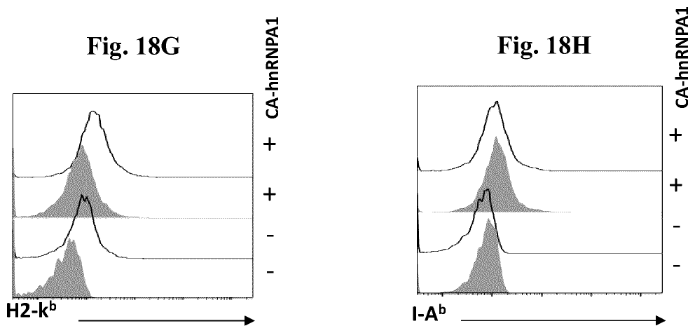

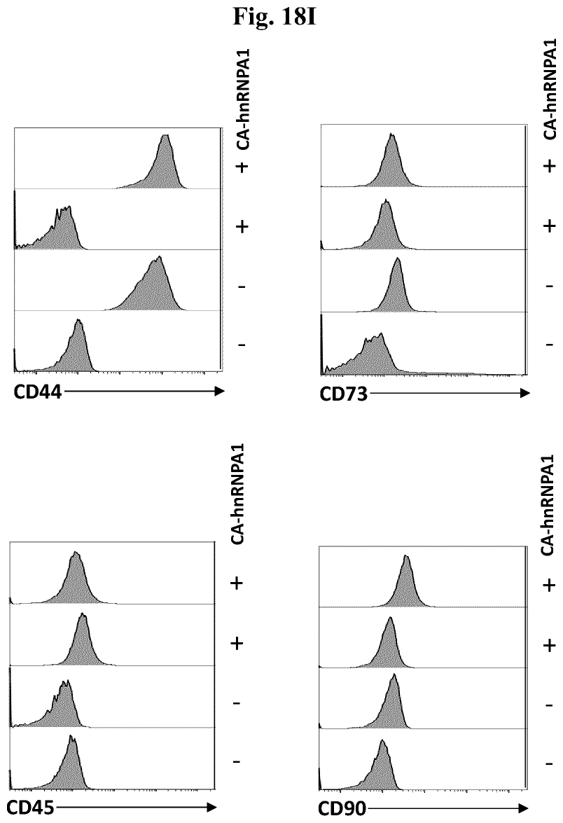

In FIG. 15, several steroid acid-peptide conjugates were shown to enhance cross presentation in an MSC cell line that was specifically engineered to express non-native cellular machinery required for cross presentation. To determine whether a similar effect occurs in wild type (WT) (i.e., non-engineered) MSCs, BM-derived MSCs (Example 1) were used instead in the same cross presentation assay with B3Z responder cells. As CA-hnRNPA1 (SEQ ID NO: 4) was shown to be amongst the best enhancers of cross presentation of OVA in the MSC cell line (FIG. 15), it was selected for subsequent studies in WT MSCs. As shown in FIG. 18A and FIG. 18B, untreated WT MSCs were capable of presenting the processed peptide (“SIINFEKL”) but poorly cross presented the complete OVA antigen (“Ova”). However, in the presence of CA-hnRNPA1, OVA cross presentation was significantly enhanced (FIG. 18A and FIG. 18B), even in comparison to CA-SV40 (FIG. 18A).

FIG. 18C shows the increase in OVA uptake by WT MSCs in the presence of CA-hnRNPA1. FIG. 18D shows the increase in OVA processing by WT MSCs in the presence of CA-hnRNPA1. FIG. 18E shows the results of the antigen cross-presentation assay conducted using different pulsing time points. FIG. 18F shows the enhancement of antigen cross-presentation assay in WT MSCs in the presence of CA-hnRNPA1 diluted in either PBS or H₂O. FIG. 18G and FIG. 18H show the increase in H2-Kb and I-Ab expression in WT MSCs treated with CA-hnRNPA1, respectively. FIG. 18I shows the phenotype characterization of CA-hnRNPA1-treated WT MSCs.

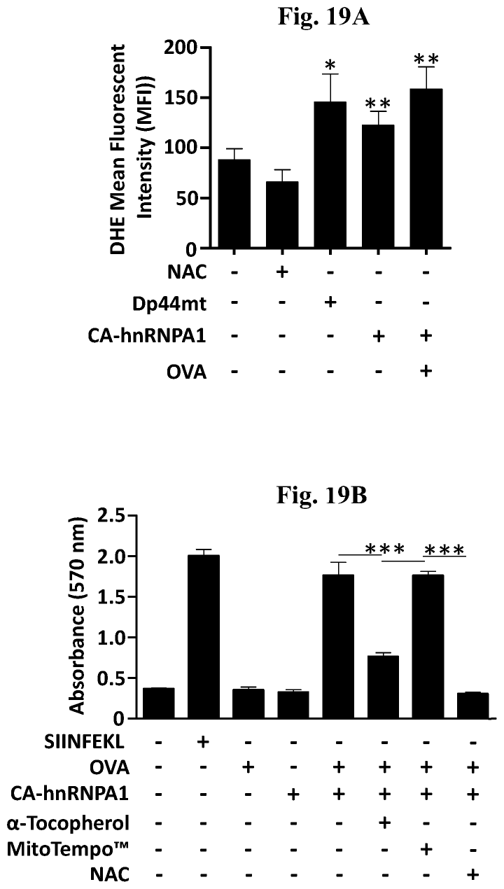

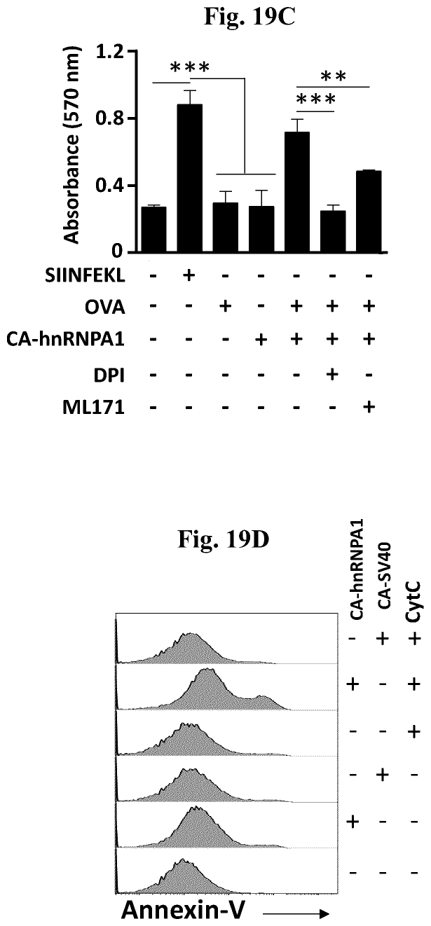

Next, the mechanism involved in cross presentation enhancement of steroid acid-peptide conjugates was further dissected. FIG. 19A-19D shows that the antigen cross-presentation capacity of WT MSCs treated with CA-hnRNPA1 requires reactive oxygen species (ROS) production. FIG. 19A shows increase in ROS production by MSCs in response to CA-hnRNPA1, as compared to Dp44mt (positive control). FIG. 19B shows the neutralization of the antigen cross-presentation capacity of CA-hnRNPA1 by α-tocopherol (lipid peroxidation inhibitor) and N-acetylcysteine (NAC; ROS inhibitor). Inhibition of mitochondrial-derived ROS, via MitoTempo™, did not neutralize the cross-presentation effect of CA-hnRNPA1. FIG. 19C shows the neutralization of the antigen cross-presentation capacity of CA-hnRNPA1 by NOX inhibitors diphenyleneiodonium chloride (DPI) and ML171. FIG. 19D shows the endosomal membrane damaging properties of CA-hnRNPA1 on MSCs co-treated with recombinant cytochrome C to increase antigen release and enhance cross-presentation.

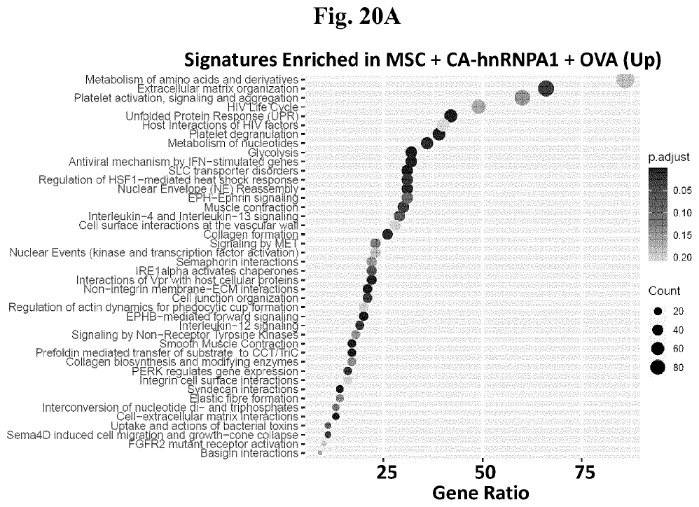

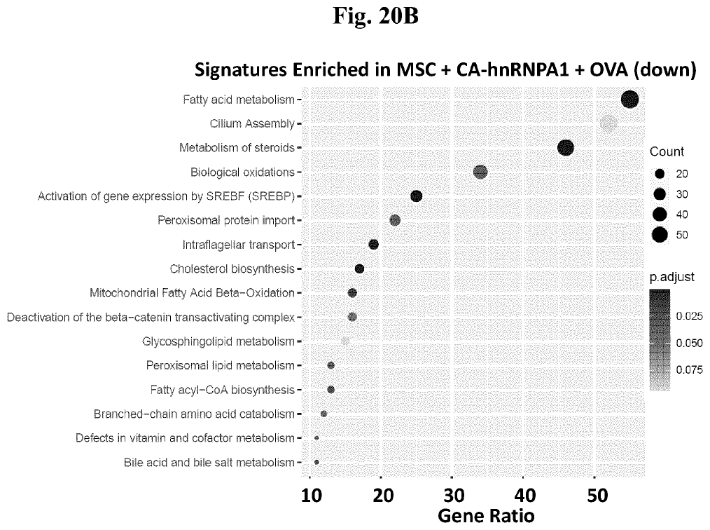

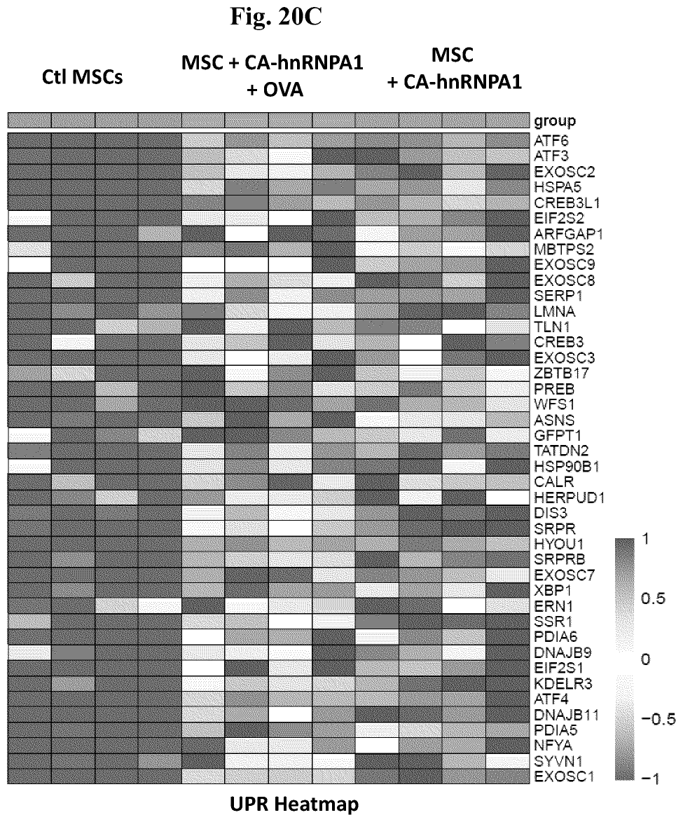

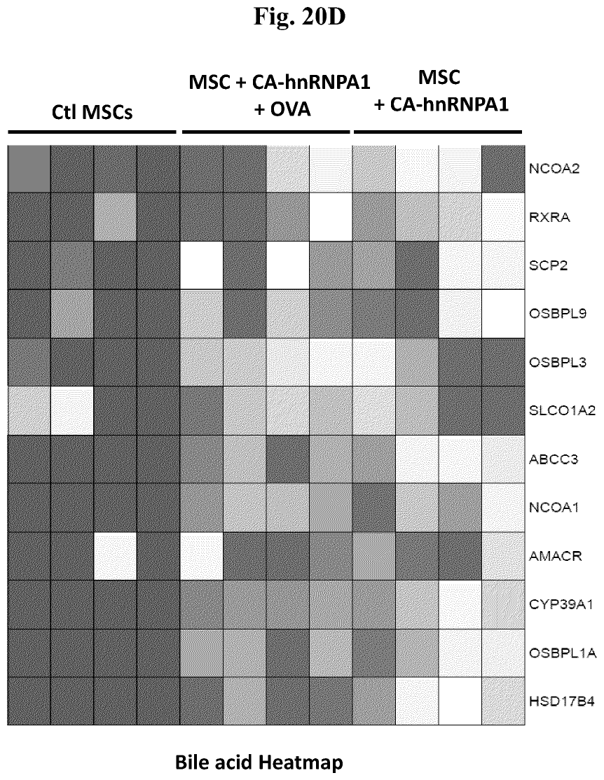

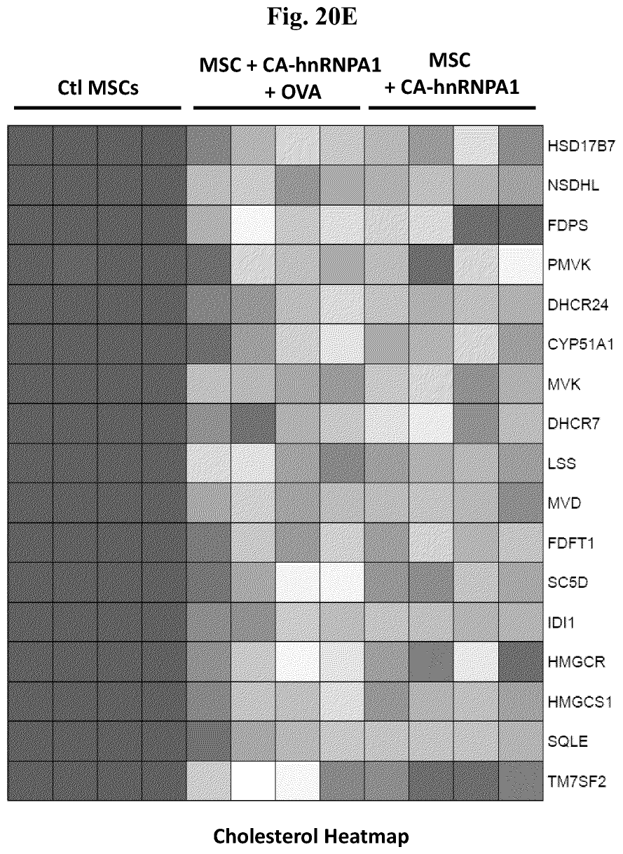

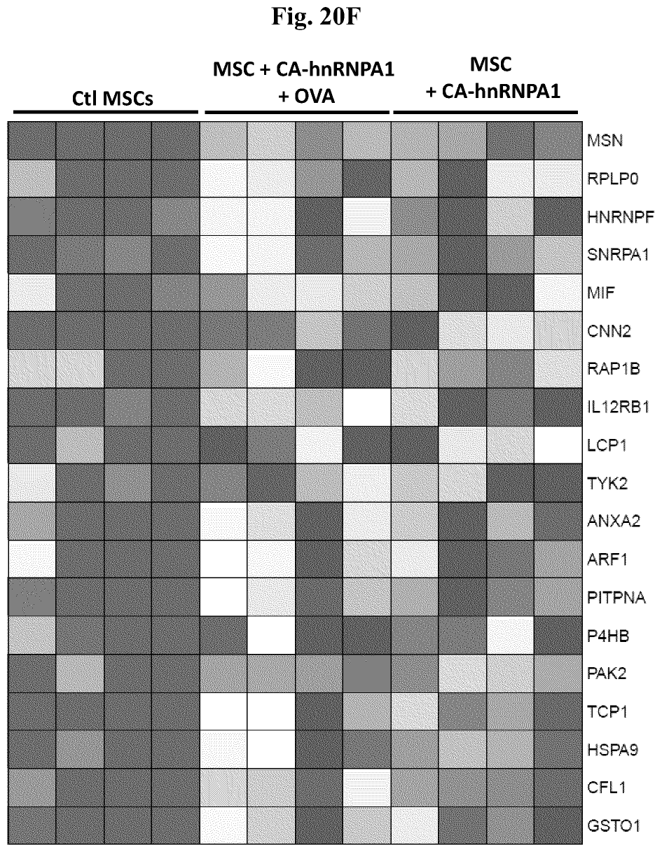

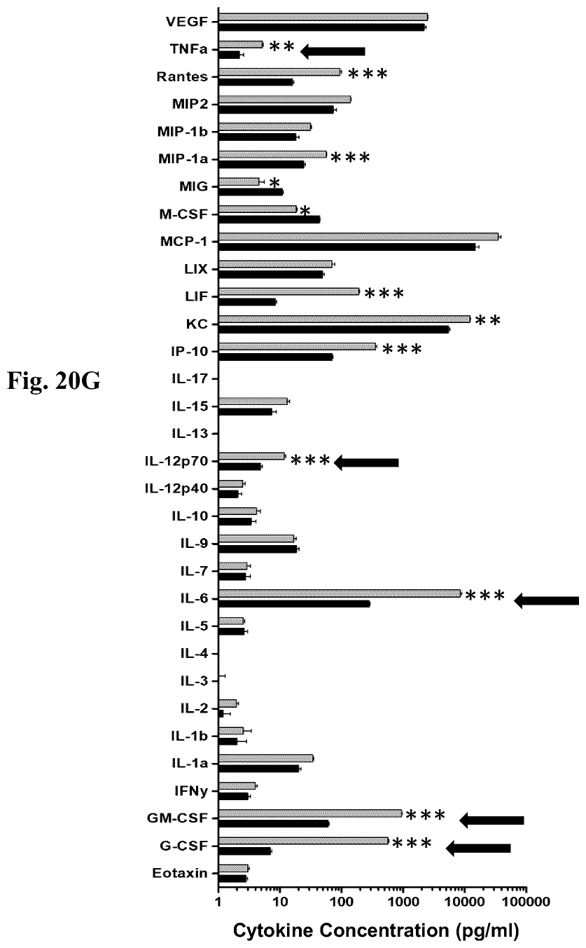

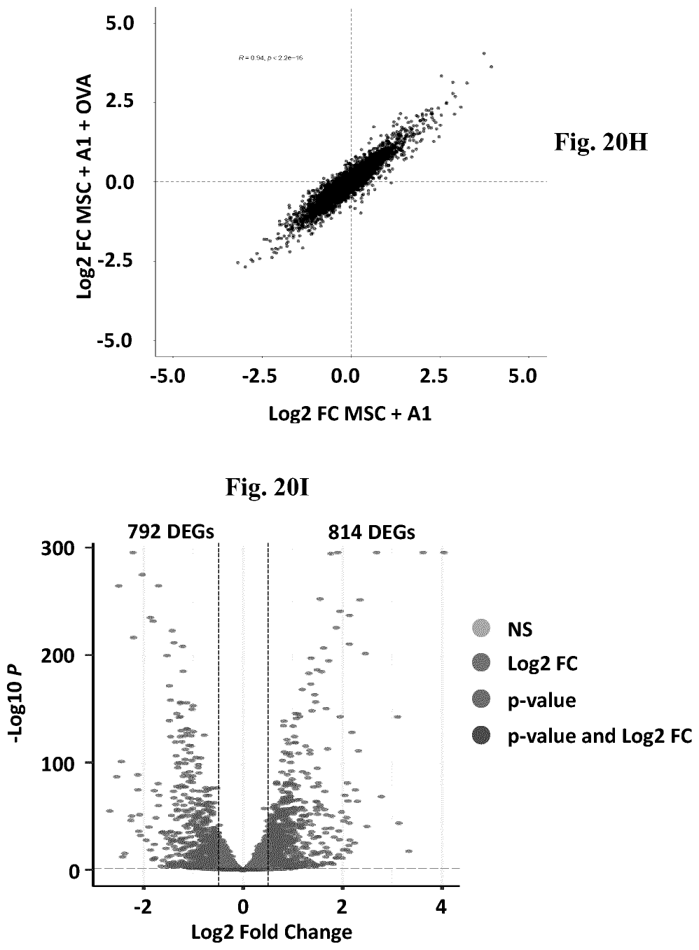

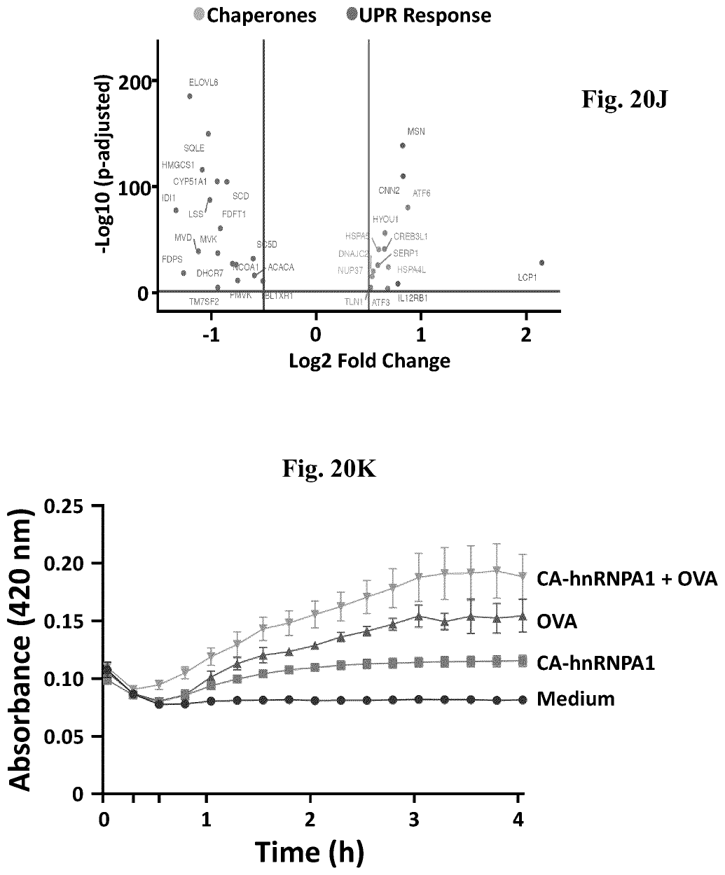

FIG. 20 shows the molecular characterization of the impact of CA-hnRNPA1 on WT MSCs. List of top reactome pathways that are enriched for both up-regulated (FIG. 20A) and down-regulated (FIG. 20B) genes in CA-hnRNPA1 treated group versus control MSCs. Coloured circles intensity corresponds to adjusted p-values; size of circles is the ratio of genes in the tested set. FIG. 20C shows a representative unfolded-protein response heatmap displaying the genes most contributing to the pathway enrichment and modulated in response to CA-hnRNPA1 treatment (FDR <5%); gene expression is scaled between −1 and +1. FIG. 20D shows the bile acid heatmap depicting genes that are modulated by CA-hnRNPA1 treatment. FIG. 20E shows the cholesterol heatmap depicting genes that are modulated by CA-hnRNPA1 treatment. Genes showing in heatmap FIG. 20D and FIG. 20E were also contributing to significant statistics form both differential expression and pathway analyses (FDR <5%). FIG. 20F shows the IL-12 heatmap depicting genes that are modulated by CA-hnRNPA1 treatment. Gene expression is scaled to -1 and 1 range. FIG. 20G shows a Luminex™ analysis showing increases in various cytokines in response to CA-hnRNPA1 treatment (in grey). For this figure, n=6/group. FIG. 20H shows the similarity of gene expression patterns between the CA-hnRNPA1 and CA-hnRNPA1+OVA groups compared to control MSCs. Correlation plot showing the spearman's rank correlation coefficient of DEGs (log 2 fold changes). FIG. 20I shows a volcano plot representing differentially expressed genes in response to CA-hnRNPA1. FIG. 20J shows a volcano plot depicting some important biological processes modulated in MSCs in response to CA-hnRNPA1. All genes from corresponding reactome analyses and showing a log 2FC greater or equal to 0.5 are labelled for further investigation. FIG. 20K shows a turbidity assay reflecting the CA-hnRNPA1 capacity to form protein aggregation mixed with the OVA protein.

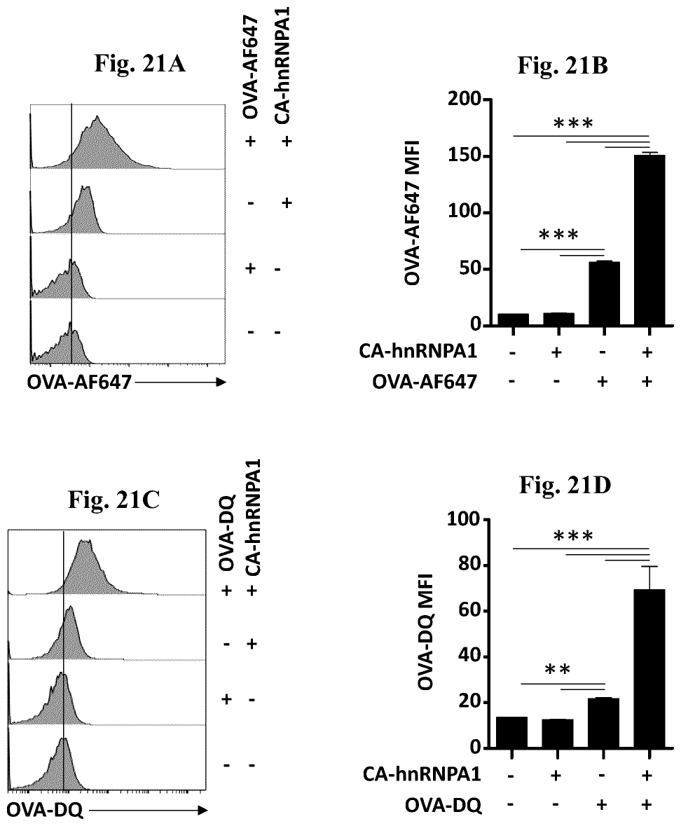

FIG. 21 shows the validation of the antigen cross-presentation properties of CA-hnRNPA1 on human WT MSCs. FIG. 21A shows the increase of OVA uptake by CA-hnRNPA1-treated human MSCs. FIG. 21B shows the signal quantification of the results presented in FIG. 21A. FIG. 21C shows the increase of OVA processing by CA-hnRNPA1-treated human MSCs. FIG. 21D shows the signal quantification of the results presented in FIG. 21C.

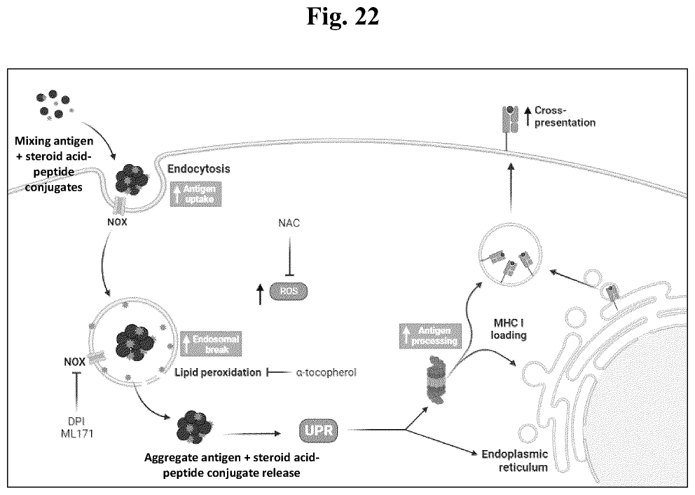

FIG. 22 shows a model of CA-hnRNPA1-mediated enhancement of antigen cross-presentation in WT human and murine MSCs. These data suggest that the mechanism involved in the enhancement of cross presentation by steroid acid-peptides involves increased endosomal ROS production, NOX activity, and lipid peroxidation, as well as increased released of aggregated antigen.

A variant of CA-hnRNPA1 in which the bile acid CA was replaced with the bile acid DCA (i.e., DCA-hnRNPA1) yielded similar results as CA-hnRNPA1 in terms of antigen cross-presentation in WT MSCs and induction of intracellular ROS.

These data demonstrate the striking enhancement of cross presentation of antigen by steroid acid-peptide conjugates in non-professional cross-presenting cells.

### Example 13: In Vivo Therapeutic Vaccination Against T-Cell Lymphoma Using an MSC-Based Vaccine Previously Pulsed with an Admixture of Antigen and Steroid Acid-Peptide Conjugates

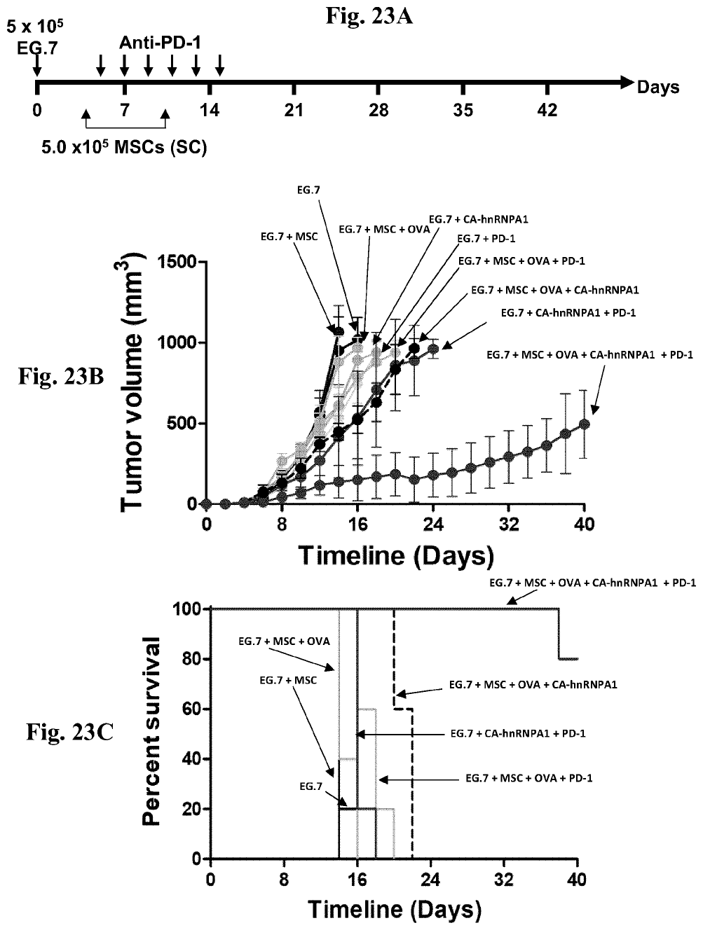

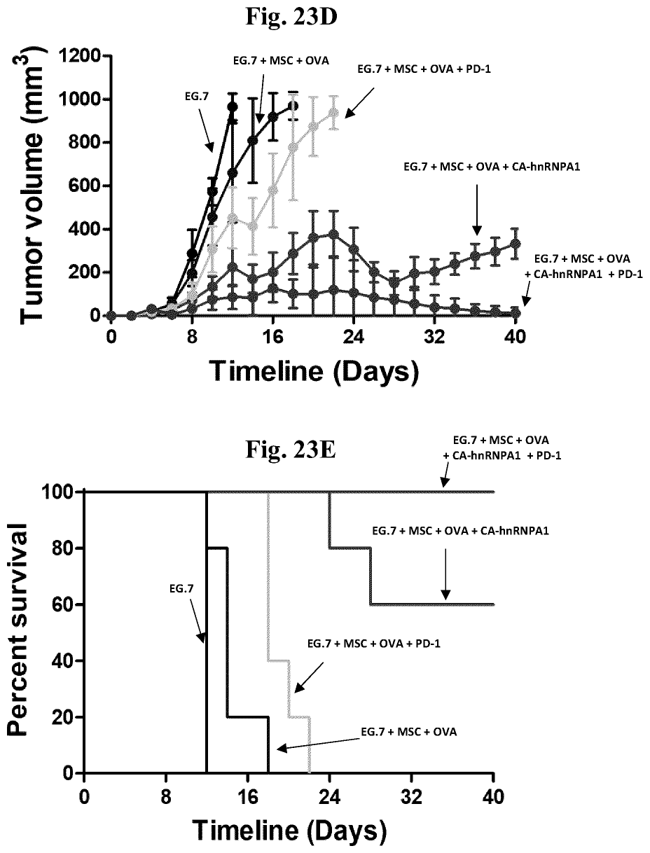

To determine the effectiveness of steroid acid peptide-conjugates in cell-based therapeutic vaccines, mice were first implanted with EG.7 lymphoma cells then immunized with WT MSCs (that were previously pulsed with OVA in the presence or absence of CA-hnRNPA1) and/or treated with the immune checkpoint inhibitor/anti-cancer agent, anti-PD-1 antibody. The immunization scheme is shown in FIG. 23A.

Mice immunized with syngeneic WT MSCs previously pulsed with OVA and CA-hnRNPA1 had significantly smaller tumors (FIG. 23B) and increased survival rates (FIG. 23C) as compared to mice immunized with anti-PD-1 antibody alone, or to MSCs pulsed with OVA alone in the presence or absence of anti-PD1 Ab. Strikingly, mice treated with a combination therapy of anti-PD-1 Ab and WT MSCs previously pulsed with OVA and CA-hnRNPA1 showed synergistic efficacy in treating T-cell lymphoma in mice, as shown by the decrease in tumor volumes and increase in survival rates.

Even stronger positive results were observed after immunization of allogeneic WT MSCs in EG.7 implanted mice, whereby mice immunized with WT MSCs previously pulsed with OVA and CA-hnRNPA1 had strikingly lower tumor volumes (FIG. 23D) and enhanced survival rates (FIG. 23E), as compared to controls. This effect was enhanced by the addition of anti-PD-1 Ab.

Overall, these findings suggest that “off-the-shelf” allogeneic or syngeneic MSCs previously pulsed with tumor antigens in the presence of steroid acid-peptide conjugates may be effectively exploited as universal vaccines to trigger potent anti-tumoral responses.

### REFERENCES

Beaudoin et al., (2016). ChAcNLS, a novel modification to antibody-conjugates permitting target cell-specific endosomal escape, localization to the nucleus and enhanced total intracellular accumulation. Molecular Pharmaceutics, 13(6): 1915-26.
Hanafi et al., (2018). Overview of Bile Acids Signaling and Perspective on the Signal of Ursodeoxycholic Acid, the Most Hydrophilic Bile Acid, in the Heart. Biomolecules, 8(4): 159.
Murakami et al., (2020). Bile acids and ceramide overcome the entry restriction for GII.3 human norovirus replication in human intestinal enteroids. Proceedings of the National Academy of Sciences USA. 117(3):1700-1710.
Shivanna et al., (2014) The crucial role of bile acids in the entry of porcine enteric calicivirus. Virology 456-457, 268-278.
Shivanna et al., (2015). Ceramide formation mediated by acid sphingomyelinase facilitates endosomal escape of caliciviruses. Virology, 483, 218-228.
Sun et al., (2016). Factors influencing the nuclear targeting ability of nuclear localization signals. Journal of Drug Targeting, 24(10): 927-933.
U.S. Pat. No. 11,291,717.

1. A composition comprising a steroid acid-peptide conjugate covalently linked to and/or admixed with a cargo for intracellular delivery.

2. The composition of claim 1, wherein the cargo is or comprises a protein, peptide, polynucleotide, polynucleotide analog, polysaccharide, drug, or any combination thereof.

3. The composition of claim 1 or 2, wherein:
(a) the cargo does not bind specifically to a cell surface receptor or ligand;
(b) the cargo is not an antibody (e.g., an antibody that binds to a cell surface epitope);
(c) the cargo is not or does not comprise an antigen; or
(d) any combination of (a) to (c).

4. The composition of any one of claims 1 to 3, wherein the cargo is or comprises a nuclease, such as a CRISPR-Cas nuclease (e.g., a class 2 CRISPR-Cas nuclease, such as Cas9 or Cas12a).

5. The composition of any one of claims 1 to 4, wherein:
(a) covalently linking the cargo to the steroid acid-peptide conjugate increases intracellular delivery and/or cytosolic/nuclear delivery of the cargo, as compared to a corresponding composition lacking the steroid acid-peptide conjugate; or
(b) the cargo is admixed with a sufficient concentration of the steroid acid-peptide conjugate to increase intracellular delivery and/or cytosolic/nuclear delivery of the cargo, as compared to a corresponding composition lacking admixture with the steroid acid-peptide conjugate.

6. The composition of any one of claims 1 to 5, wherein the cargo is covalently linked to a sufficient number of steroid acid-peptide moieties such that the cargo exhibits greater stability (e.g., thermal stability) than the unmodified cargo.

7. The composition of any one of claims 1 to 6, wherein the steroid acid is or comprises a bile acid (e.g., a primary bile acid or a secondary bile acid).

8. The composition of any one of claims 1 to 7, wherein the steroid acid is or comprises:
(a) a bile acid which is: cholic acid (CA), chenodeoxycholic acid (CDCA), deoxycholic acid (DCA), lithocholic acid (LCA), glycodeoxycholic acid (GDCA), glycocholic acid (GCA), taurocholic acid (TCA), glycodeoxycholic acid (CDCA), glycochenodeoxycholic acid (GCDCA), taurodeoxycholic acid (TDCA), glycolithocholic acid (GLCA), taurolithocholic acid (TLCA), taurohyodeoxycholic acid (THDCA), taurochenodeoxycholic acid (TCDCA), ursocholic acid (UCA), tauroursodeoxycholic acid (TUDCA), ursodeoxycholic acid (UDCA), or glycoursodeoxycholic acid (GUDCA);
(b) an analog of the bile acid of (a) that: induces endocytosis; triggers ceramide accumulation on the inner leaflet of endosomes; triggers increased acid sphingomyelinase (ASM)-mediated cleavage of sphingomyelin to form ceramide; and/or has a hydrophobicity greater than that of cholic acid;
(c) a bile acid or bile acid analog that is more hydrophobic than cholic acid (e.g. CDCA, DCA, LCA, TCA, TDCA, TCDCA, GCA, GDCA, or GCDCA); or
(d) any combination of (a) to (c).

9. The composition of any one of claims 1 to 8, wherein each cargo molecule is covalently linked to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 steroid acid-peptide moieties.

10. The composition of any one of claims 1 to 9, wherein the steroid acid-peptide conjugate is covalently linked to the cargo via a cleavable or non-cleavable linker (e.g., bifunctional, trifunctional linker, or multi-functional linker).

11. The composition of any one of claims 1 to 10, wherein the steroid acid-peptide conjugate is covalently linked to the cargo via said peptide (e.g., the steroid acid is conjugated at or towards the N- or C-terminus of the peptide).

12. The composition of any one of claims 1 to 11, wherein the peptide:
(i) comprises a protein transduction domain that stimulates endocytosis and/or endosomal formation;
(ii) comprises a subcellular targeting signal;
(iii) is a cationic peptide (e.g., a non-cell-penetrating cationic peptide);
(iv) is a non-immunogenic peptide;
(v) comprises at least one cysteine residue (e.g., at or towards the peptide's N- and/or C terminus) having a free thiol group or a thiol group that is protected in a cleavable manner (e.g., by a pharmaceutically acceptable protecting group); or
(vi) any combination of (i) to (v).

13. The composition of any one of claims 1 to 12, wherein: the steroid acid is not or does not comprise cholic acid; the NLS peptide is not or does not comprise an SV40 NLS; and/or the steroid acid-peptide conjugate is not or does not comprise CA-SV40.

14. The composition of any one of claims 1 to 13, wherein the peptide is or comprises a nuclear localization signal which is a classical NLS (e.g., NLS from SV-40 large T-antigen (e.g., PKKKRKV; SEQ ID NO: 1 or 2) or from other classical NLSs) or a non-classical NLS (e.g., acidic M9 domain in the hnRNP A1 protein; the sequence KIPIK in yeast transcription repressor Matα2; PY-NLS; ribosomal NLS; and the complex signals of U snRNPs).

15. The composition of any one of claim 1 to 14, wherein the peptide is or comprises a nuclear localization signal which is a/an: SV40 NLS (e.g., comprised in SEQ ID NO: 1 or 2), GWG-SV40NLS (e.g., comprised in SEQ ID NO: 3), hnRNPA1 M9 NLS (e.g., comprised in SEQ ID NO: 4), hnRNP D NLS (e.g., comprised in SEQ ID NO: 5), hnRNP M NLS (e.g., comprised in SEQ ID NO: 6), PQBP-1 NLS (e.g., comprised in SEQ ID NO: 7), NLS2-RG Domain RPS17 (e.g., comprised in SEQ ID NO: 8), NLS1 RPS17 (e.g., comprised in SEQ ID NO: 9), NLS2 RPS17 (e.g., comprised in SEQ ID NO: 10), NLS3 RPS17 (e.g., comprised in SEQ ID NO: 11), cMyc NLS (e.g., comprised in SEQ ID NO: 12), HuR NLS (e.g., comprised in SEQ ID NO: 13), Tus NLS (e.g., comprised in SEQ ID NO: 14), or Nucleoplasmin NLS (e.g., comprised in SEQ ID NO: 15), or is a variant of an NLS having nuclear localization activity, the NLS comprising or consisting of the amino acid sequence of any one of SEQ ID NOs: 1 to 15.

16. The composition of any one of claims 1 to 15, wherein the steroid acid comprises CA or DCA, and the peptide comprises an hnRNPA1 M9 NLS or a variant thereof having nuclear localization activity.

17. The composition of any one of claim 1 to 16, wherein the peptide does not comprise an endosomal escape motif, or protein transduction motif, or cell penetrating motif.

18. The composition of any one of claims 1 to 17, wherein the composition or conjugate is formulated within a hydrogel, liposome, lipid-based transfection agent, or nanoparticle (e.g., lipid nanoparticle).

19. The composition of any one of claims 1 to 18, further comprising pharmaceutically or physiologically acceptable carrier and/or excipient.

20. The composition of any one of claims 1 to 19, for use in:
(a) increasing the intracellular, cytosolic, and/or nuclear delivery of a biologically active cargo (e.g., therapeutic cargo or diagnostic cargo) in vitro or in vivo, as compared to a corresponding composition lacking the steroid acid-peptide conjugate;
(b) increasing presentation of an antigenic polypeptide cargo by target cells, such as by professional anti-presenting cells (e.g., dendritic cells, macrophages, B cells, or non-immune cells engineered for overexpression of an immunoproteasome), or by non-professional antigen-presenting cells (e.g., wild-type, engineered, primary, and/or cultured non-immune cells, such as mesenchymal stromal cells (MCSs));
(c) increasing intracellular reactive oxygen species production in target cells, such as by professional anti-presenting cells (e.g., dendritic cells, macrophages, B cells, or non-immune cells engineered for overexpression of an immunoproteasome), or by non-professional antigen-presenting cells (e.g., wild-type, engineered, primary, and/or cultured non-immune cells, such as MCSs);
(d) transforming immunosuppressive cells (e.g., immunosuppressive MSCs) into immunostimulatory and/or proinflammatory MSCs; or
(e) any combination of (a) to (d).

21. The composition for use of claim 20, wherein the composition is adapted or formulated for oral, intravenous, intranasal, intramuscular, subcutaneous, intradermal, intratumoral, intracranial, topical, intrarectal administration, or any other route of administration.

22. A method for delivering a cargo intracellularly, the method comprising providing a composition as defined in any one of claims 1 to 21, and administering the composition to target cells in vitro or in vivo.

23. A method for preparing a cargo for intracellular delivery having increased stability, the method comprising covalently linking the cargo to a sufficient number of steroid acid-peptide moieties to produce a covalently-modified cargo that exhibits greater stability (e.g., thermal stability) than the corresponding unmodified cargo.

24. The method of claim 23, wherein the cargo and/or the steroid acid-peptide is as defined in any one of claims 1 to 17.

25. The method of claim 23 or 24, wherein the cargo is reacted or admixed with between a 2-fold and 1000-fold, 2-fold and 500-fold, 2-fold and 200-fold, 2-fold and 100-fold molar excess of the steroid acid-peptide conjugate; between a 2-fold and 50-fold molar excess of the steroid acid-peptide conjugate; or between a 5-fold and 25-fold molar excess of the steroid acid-peptide conjugate.

26. The method of any one of claims 23 to 25, wherein the mean number of steroid acid-peptide moieties conjugated per cargo, or the molar ratio of cargo: steroid acid-peptide conjugate admixed, is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50; or wherein the mean number of steroid acid-peptide moieties conjugated per cargo is between about 1, 2, 3, 4, 5, 6, 7, 8, 9, or 10 and n, wherein n is the total number of accessible sites on the cargo available for conjugation.

27. A composition comprising an antigen covalently linked to and/or admixed with a steroid acid-peptide conjugate in an amount sufficient to improve presentation of the antigen upon administration of the composition to non-antigen presenting cells (e.g., mesenchymal stromal cells [MSCs]), as compared to administration of a corresponding composition lacking the steroid acid-peptide conjugate.

28. The composition of claim 27, wherein the steroid acid or peptide is as defined in any one of claims 7, 8, or 10 to 17.

29. The composition of claim 27 or 28, wherein the molar ratio of steroid acid-peptide conjugate to antigen in the composition is at least 0.01:1, 0.05:1, 0.1:1, 0.2:1, 0.5:1, 1:1, 2:1, 3:1, 4:1, 5:1, 6:1, 7:1, 8:1, 9:1, 10:1, 15:1, 20:1; is no more than 2:1, 3:1, 4:1, 5:1, 6:1, 7:1, 8:1, 9:1, 10:1, 15:1, 20:1, 50:1, 100:1, 250:1, 500:1, 1000:1, and/or is between 1:1 to 1000:1; 1:1 to 500:1, 1:1 to 250:1, 1:1 to 200:1.

30. The composition of any one of claims 27 to 29, wherein the steroid acid is conjugated to the peptide:
(a) at a molar ratio of steroid acid: peptide of 1:1, 2:1, 3:1, 4:1, 5:1, 6:1, 7:1, 8:1, 9:1, or 10:1, or between 1:1 to 10:1;
(b) at a free amino group and/or a free thiol group (e.g., of a lysine or cysteine) of the peptide;
(c) at or towards the N-terminal end of the peptide (e.g., at the free amino group of N terminal residue and/or at the thiol group of an N-terminal cysteine residue); or
(d) any combination of (a) to (c).

31. The composition of any one of claims 27 to 30, wherein the antigen is a polypeptide antigen comprising one or more MHC class I epitopes and/or MHC class II epitopes

32. The composition of any one of claims 27 to 31, wherein the antigen is or comprises:
(a) a tumor-associated antigen (TAA), tumor-specific antigen (TSA), tumor lysate, a neoantigen, a viral antigen, a bacterial antigen, a fungal antigen, an antigen associated with a disease or disorder amenable to treatment by vaccination and/or immunotherapy; or any antigenic fragment thereof; or
(b) a corona viral antigen (e.g., SARS-CoV-2 Spike protein, SARS-CoV Spike protein, or an antigenic fragment thereof; or a cancer antigen, such as a single-nucleotide variant antigen, a mutational frameshift antigen, splice variant antigen, a gene fusion antigen, an endogenous retroelement antigen, or another class of antigen, such as a human leukocyte antigen (HLA)-somatic mutation-derived antigen or a post-translational TSA, a viral-derived cancer antigen (e.g., from human papillomavirus (HPV), cytomegalovirus, or Epstein-Barr virus (EBV)), a cancer-testis antigen, HER2, PSA, TRP-1, TRP-2, EpCAM, GPC3, CEA, MUC1, MAGE-A1, NY-ESO-1, SSX-2, mesothelin (MSLN), EGFR, cell lysates or other material derived from a tumor (e.g., tumor-derived exosomes).

33. The composition of any one of claims 27 to 32, further comprising a pharmaceutically acceptable excipient and/or adjuvant.

34. A cell culture comprising non-antigen presenting cells (e.g., mesenchymal stromal cells [MSCs]) and the composition as defined in any one of claims 27 to 33.

35. A cell culture comprising non-antigen presenting cells (e.g., mesenchymal stromal cells [MSCs]) pulsed with an antigen covalently linked to and/or admixed with a steroid acid-peptide conjugate.

36. A vaccine comprising the composition as defined in any one of claims 27 to 32, or comprising cells produced using the cell culture as defined in claim 34 or 35.

37. The vaccine of claim 36, which is a therapeutic or prophylactic vaccine (e.g., anti-cancer vaccine, anti-viral vaccine, or anti-bacterial vaccine).

38. A method for enhancing presentation of an antigen of interest in a subject or cells, the method comprising administering to the subject or in non-antigen presenting cells (e.g., mesenchymal stromal cells [MSCs]) the composition as defined in any one of claims 27 to 33, or cells produced using the cell culture as defined in claim 34 or 35.

39. A method for vaccinating a subject against an infectious disease, the method comprising administering to the subject the composition as defined in any one of claims 27 to 33 or cells produced using the cell culture as defined in claim 34 or 35, wherein the antigen comprises an antigenic fragment of a pathogen (e.g., virus, bacteria, fungus) causing the infectious disease.

40. A method for treating cancer in a subject, the method comprising administering to the subject the composition as defined in any one of claims 27 to 33 or cells produced using the cell culture as defined in claim 34 or 35, wherein the antigen is an overexpressed or aberrantly expressed in cells causing the cancer.

41. The composition as defined in any one of claims 27 to 33, or the cell culture as defined in claim 34 or 35, for use in:
(i) generating enhancing presentation of an antigen of interest in a subject or in non-professional antigen presenting cells (e.g., mesenchymal stromal cells [MSCs]);
(ii) the manufacture of a medicament (e.g., vaccine) for generating an immune response in a subject;
(iii) increasing presentation of an antigenic polypeptide cargo by non-professional antigen-presenting cells (e.g., wild-type, engineered, primary, and/or cultured non-immune cells, such as mesenchymal stromal cells [MCSs]);
(iv) increasing intracellular reactive oxygen species production in by non-professional antigen-presenting cells (e.g., wild-type, engineered, primary, and/or cultured non-immune cells, such as MCSs);
(v) transforming immunosuppressive cells (e.g., immunosuppressive MSCs) into immunostimulatory and/or proinflammatory MSCs; or
(vi) any combination of (i) to (v).
